(19) United States
(12) Patent Application Publication
(10) Pub. No.: US 2019/0189290 A1
(43) Pub. Date: Jun. 20, 2019

(54) METHOD AND SYSTEM FOR MEDICAL CANNABIS EDUCATION AND OPERATION, AND PROMOTION AND FACILITATION OF COMMUNICATION AND COMMERCE BETWEEN MEDICAL CANNABIS PATIENTS, PHYSICIANS, CULTIVATORS, PROCESSORS, SELLERS AND OTHERS

(71) Applicant: Healthy Option Consulting Inc.

(72) Inventor: Andrew Farber, Boca Raton, FL (US)

(21) Appl. No.: 16/203,158

(22) Filed: Nov. 28, 2018

Related U.S. Application Data
(60) Provisional application No. 62/649,674, filed on Mar. 29, 2018.
(60) Provisional application No. 62/599,812, filed on Dec. 18, 2017.

(51) Int. Cl. G16H 70/40; G16H 10/60; G16H 80/00; G16H 40/20

(52) U.S. Cl. CPC G16H 70/40; G16H 10/60; G09B 23/28; G16H 40/20; G16H 80/00

(57) ABSTRACT

Provided is a system and method for educating medical practitioners as to why they should recommend the use of medical cannabis to their patients, how they can incorporate medical cannabis into their practices, how they can recommend the use of medical cannabis to their patients safely, efficiently, profitably, legally and without fear of professional liability, how patients can be directed to their practices, how the owner of the method or system can: i)consult with the medical practitioners to build and design a medical cannabis practice for them and provide initial operational training for them; and ii) jointly develop, own and operate a medical cannabis practice that is managed by the owner of the method or system. Also provided is a system and method for educating patients as to the medical benefits of cannabis, how they can qualify for their Medical Marijuana ID Cards, and how they can be referred to certain physicians and dispensaries. Further provided is a system and method for cannabis product growers, processors and sellers with access to the system to communicate directly with physicians and patients having access to the system and facilitate business with them. Also provided is a system and method for lawyers, insurers, electronic medical record service providers, and other vendors with access to the system to market their goods or services to medical cannabis practitioners, medical cannabis patients, and cannabis product growers, processors and dispensers. Also provided is a system to provide a database of all information relating to medical cannabis.

### FIELD OF THE INVENTION

The invention relates to a system and method for educating medical practitioners as to why they should recommend the use of medical cannabis to their patients, how they can incorporate medical cannabis into their practices, how they can recommend the use of medical cannabis to their patients safely, efficiently, profitably, legally and without fear of professional liability, how they can have the owner of the present invention (the “Owner”) design and build a medical cannabis practice for the medical practitioners and train medical practitioners and their staff how to operate the practice on their own, and how they can jointly develop, own and operate a medical cannabis practice with the Owner, and how medical cannabis patients can be directed to their practice. The invention further relates to forming a HIPAA compliant patient database that includes pertinent patient information regarding the patient's medical cannabis experiences. The invention further relates to a system and method for educating patients about the therapeutic effects of medical cannabis, removing any stigma regarding cannabis use, and directing patients to certain physicians and dispensaries utilizing the invention. The invention further relates to providing cannabis growers, processors and sellers with direct access to the population of medical practitioners who may recommend the use of their products to their patients and to the medical cannabis patients who could be using their products. The invention further relates to providing medical practitioners recommending the use of medical cannabis and medical cannabis patients with access to cannabis related legal and/or compliance advice from the Owner, lawyers and others, keeping them up to date with the latest technology and law, providing them with a database of cannabis related medical research and other information, access to one another so that they may ask questions of one another and/or share their respective experiences and successes. The invention also relates to providing lawyers, insurers and others with access to the system so that they may provide legal advice, insurance services, and other goods or services to medical cannabis practitioners, medical cannabis patients and cannabis product growers, processors and dispensers.

### BACKGROUND OF THE INVENTION

Cannabis, also known as marijuana, is a genus of flowering plant in the Cannabaceae family. It is known to have three species, Cannabis Indica, Cannabis Sativa and Cannabis Ruderalis, the first two of which are commonly found in North America. The Cannabis Sativa and Cannabis Indica strains, as well as their many constituent cannabinoids, are known to have therapeutic effects on human physiology by acting on the human endocannabinoid system.

Cannabis and its constituent cannabinoids are classified as Schedule 1 controlled substances under the US Controlled Substances Act (“CSA”) and are illegal to grow, possess and sell under federal law. However, it is legal (at the state, local or territorial level) for the treatment of a variety of chronic medical conditions in 30 states, Guam, Puerto Rica and the District of Columbia. These jurisdictions have enacted legislation and/or regulations pursuant to which medical cannabis can be purchased or grown by patients after their chronic conditions have been certified by a physician and the physician has recommended the use of medical cannabis for the treatment of such conditions.

Given its status as a Schedule 1 controlled substance under the CSA, there is little acceptable medical research concerning medical cannabis and its therapeutic effects. As it has been historically grown and sold through the black market with no meaningful quality control protocols, it has not been conventionally viewed as a medicine and there is little information available concerning appropriate dosing regimens, reported side effects and contraindications. These are the most common shortcomings cited by physicians who have been asked why they do not recommend the use of medical cannabis to their patients.

Similarly, the population at large has been conditioned to view cannabis as an illegal substance with no redeeming or therapeutic effects. They are generally unaware of the true therapeutic effects of cannabis and are reluctant to consider its use given the stigma of its controlled substance classification under the CSA.

Thus, there is a need for a system to: i) let physicians know why they should be recommending the use of medical cannabis to their patients; ii) show physicians how to incorporate medical cannabis into their practices; iii) provide physicians with the information that they need to make their medical cannabis recommendations safely, efficiently, profitably, legally and with little risk of professional liability; iv) show physicians how medical cannabis patients can be directed to their practices; v) educate patients regarding the different strains of medical cannabis, their constituent compounds and their respective therapeutic effects; vi) let patients know how they can qualify for their Medical Marijuana ID Card; vii) identify to patients those physicians in their community possessing superior medical cannabis product, practice management, and regulatory knowledge who conduct their medical cannabis practices in accordance with the highest professional standards; viii) show patients where they can purchase their medical cannabis and delivery devices; ix) provide patients and physicians with direct branding and other messaging information from growers, processors and/or dispensaries; and x) provide medical cannabis physicians, patients and cannabis product growers, processors and dispensers with access to venders of legal, insurance, creditor-proofing, practice management and other goods and services to receive preferred pricing or other benefits.

### SUMMARY OF THE INVENTION

An objective of the invention is to provide a method and system to educate medical practitioners as to why they should recommend the use of medical cannabis to their patients and how they can incorporate it into their practices. Medical practitioners can join as physician members to learn how to make their medical cannabis recommendations safely, profitably, efficiently, legally and without fear of professional liability and find out how we can direct medical cannabis patients to their practices.

Another objective of the Invention is to facilitate the sale of consulting services to medical practitioners and to design and build a medical cannabis practice for medical practitioners and/or train the medical practitioners and their staff how to operate the practice.

Another objective of the invention is to co-venture with medical practitioners in connection with the development, opening and operation of a medical cannabis practice to be co-owned by the physician and the Owner, whereby the Owner is responsible for the operations and management of the medical cannabis practice with the assistance of the method and system. The Owner includes individual(s), partnership(s), and/or corporation(s) or other entities having any amount or type of ownership in the present invention, including a license, or a indirect or direct ownership interest in the invention.

Another objective of the invention is to provide information to patients regarding medical cannabis, medical practitioners who make recommendations of medical cannabis in their respective communities, and dispensaries where they can purchase medical cannabis and delivery devices.

A further objective of the invention is to form a patient database that includes pertinent HIPAA compliant patient information regarding the patient's medical cannabis experiences. The patient database can include information that would ordinarily be found in a patient's medical cannabis record, including but not limited to information relating to the condition(s) for which medical cannabis is recommended, dosages applied, side effects, drug interactions, the effects of medical cannabis use on opioid use and/or addiction, other medications being taken by the patient, other medical conditions that the patient is suffering from, and efficacy of medical cannabis for the treatment of the patient's chronic condition(s). The patient database can be used for marketing and research.

Another objective of the invention is to provide medical cannabis growers, processors and sellers with direct access to the population of medical practitioners who may recommend the use of their products to their patients and to the medical cannabis patients who could be using their products so that they may communicate and do business with one another. The invention further relates to providing medical practitioners recommending the use of medical cannabis and medical cannabis patients with access to cannabis related legal and/or compliance related information from the Owner and others, keeping them up to date with the latest technology and law, providing them with a database of medical cannabis related research and other information, access to other physicians, patients or others so that they may ask questions of one another and/or share their respective experiences and successes.

A further objective of the invention is to provide lawyers, insurers, practice management product vendors and others with access to the system so that they may market their products or services to medical cannabis practitioners, medical cannabis patients, and cannabis product growers, processors and dispensers, and to provide a database of all information relating to medical cannabis.

A further objective of the invention is to provide a HIPAA complaint database of all patient related medical cannabis information and a database of all other medical cannabis related information.

The system and method can be utilized in states that permit recreational cannabis use, in which case the patients/users interact directly with the dispensaries and optionally with physicians. The system and method can be utilized in states that do not permit recreational use and require a physician to recommend cannabis, in which case the patients interact with the physicians and dispensaries. The system and method can ensure that cannabis can be supplied to a user in compliance with state and local laws and rule, and if legalized at a national level, in compliance with federal law.

The system and method can act as a clearinghouse, marketplace, and exchange between patients, individuals, physicians, legal growers, legal processors and legal retailers of cannabis, any two of them, and/or those people doing business with them, to facilitate communication and commerce between any number of them.

These objectives and other objectives can be obtained by a computer-implemented method for providing information relating to medical cannabis comprising:
connecting, by a user, a user interface device over a network to a web interface of a cannabis website stored on a server connected to the network; and
sending information, by the server, to the user interface device, the information including information relating to medical cannabis.

These objectives can also be obtained by a computer printable medium storing instructions in a non-volatile memory executable by the computer, wherein execution in the instructions implements the method for providing information relating to cannabis.

These objectives and other objectives can also be obtained by providing a patient database, a cannabis legal database, a medical cannabis research database, a cannabis product cultivator, processor and/or dispensary database, a medical practitioner database, and/or a cannabis banking database.

The objectives of the invention and other objectives can also be obtained by a computer-implemented method for providing medical cannabis comprising:
providing a system comprising a server having a processor and a non-transitory tangible memory in communication with the processor, the server is connected to a network, the server is configured to provide a web interface on a user interface device in communication with the server over the network, and the memory having stored therein a member medical practitioner database, member patient database, member dispensary database, medical practitioner information, patient information, and dispensary information;
   the member medical practitioner database comprising a list of medical practitioners that are members of the system and contact information;
   the member patient database comprising a list of the patients that are members of the system, contact information, and medical information;
   the member dispensary database comprising a list of dispensaries that are members of the system, their contact information, and product information;
   the medical practitioner information comprising at least one of cannabis education for the medical practitioner as to why the medical practitioner should recommend use of medical cannabis to a patient, how the medical practitioner can incorporate medical cannabis into a practice, how the medical practitioner can recommend use of medical cannabis safely, profitably, legally and without fear of professional liability, and how prospective medical cannabis patients can be directed to the medical practice,
   the patient information comprising at least one of information relating to medical cannabis education and uses, dosing, administration instructions, and how a prospective patient can qualify to use medical cannabis, and
   the dispensary information comprising name and contact information of a dispensary as well as a description and the availability of its products,
connecting, by a medical practitioner, a first user interface device over the network to the server so that the web interface is displayed on the first interface device;
sending the medical practitioner information, by the server, to the first user interface device;
connecting, by a patient, a second user interface device over the network to the server so that the web interface is displayed on the second user interface device;
sending patient information, by the server, to the second user interface device;
connecting, by a dispensary, a third user interface device over the network to the server so that the web interface is displayed on the third user interface device;
sending dispensary information by the server, to the first and second user interface devices; and
the patient receiving cannabis from a dispensary in compliance with state law.

The objectives of the invention and other objectives can further be obtained by a system for providing information relating to cannabis comprising:
a server having a processor and a non-transitory tangible memory in communication with the processor, the server is connected to a network, the server is configured to provide a web interface on a user interface device in communication with the server over the network, and the memory having stored therein a member medical practitioner database, member patient database, member dispensary database, medical practitioner information, patient information, and dispensary information;
   the member medical practitioner database comprising a list of medical practitioners that are members of the system and contact information;
   the member patient database comprising a list of the patients that are members of the system, contact information, and medical information;
   the member dispensary database comprising a list of dispensaries that are members of the system, their contact information, and product information;
   the medical practitioner information comprising at least one of cannabis education for the medical practitioner as to why the medical practitioner should recommend use of medical cannabis to a patient, how the medical practitioner can incorporate medical cannabis into a practice, how the medical practitioner can recommend use of medical cannabis safely, profitably, legally and without fear of professional liability, and how prospective medical cannabis patients can be directed to the medical practice,
   the patient information comprising at least one of information relating to medical cannabis education and uses, dosing, administration instructions, and how a prospective patient can qualify to use medical cannabis, and
   the dispensary information comprising name and contact information of a dispensary as well as a description and the availability of its products; and a user interface device constructed to connect to the server over the network.

Various aspects of the described example embodiments can be combined with aspects of certain other example embodiments to realize yet further embodiments. It is to be understood that one or more features of any one example can be combined with one or more features of the other example. In addition, any single feature or combination of features in any example or examples can constitute patentable subject matter. Other features of the technology will be apparent from consideration of the information contained in the following detailed description.

### BRIEF DESCRIPTION OF THE DRAWINGS

The following Detailed Description can be better understood when read in conjunction with the appended drawings. For the purposes of illustration, the drawings show example embodiments, but the subject matter is not limited to the specific elements and instrumentalities disclosed.

FIG. 1 illustrates an exemplary system.

FIG. 2A is an example data flow of the free physician front end of the present invention.

FIG. 2B is an example data flow of the paid for physician back end of the present invention.

FIG. 2C is an example of a flow chart of the free patient front end of the present invention.

FIG. 2D is an example of a flow chart of the paid for patient back end of the present invention.

FIG. 3 is a screen shot of an exemplary physician Home page.

FIG. 4 is a screen shot of an exemplary About Us page.

FIG. 5A is a screen shot of the exemplary Why You Should Recommend the Use of Medical Marijuana to Your Patients page, Expand your practice.

FIG. 5B is a screen shot of the exemplary Why You Should Recommend the Use of Medical Marijuana to Your Patients page, Improve the lives of your patients.

FIG. 5C is a screen shot of the exemplary Why You Should Recommend the Use of Medical Marijuana to Your Patients page, Convenient access to peer reviewed and anecdotal research.

FIG. 5D is a screen shot of the exemplary Why You Should Recommend the Use of Medical Marijuana to Your Patients page, Medical Marijuana Treatment Center Product Knowledge.

FIG. 5E is a screen shot of the exemplary Why You Should Recommend the Use of Medical Marijuana to Your Patients page, Patient initiatives.

FIG. 5F is a screen shot of the exemplary Why You Should Recommend the Use of Medical Marijuana to Your Patients page, Rules and regulations.

FIG. 5G is a screen shot of the exemplary Why You Should Recommend the Use of Medical Marijuana to Your Patients page, Physician forum.

FIG. 5H is a screen shot of the exemplary Why You Should Recommend the Use of Medical Marijuana to Your Patients page, Upcoming events.

FIG. 6 is a screenshot of an exemplary Join Our Physician Network page.

FIG. 7 is a screenshot of an exemplary Site Directory page for the physician portal portion of the website.

FIG. 8 is a screenshot of an exemplary Peer Reviewed and Anecdotal Medical Cannabis Research page for the physician portal portion of the website.

FIG. 9 is a screenshot of an exemplary Medical Marijuana Treatment Center Product Knowledge page.

FIG. 10 is a screenshot of an exemplary Our Patient Initiatives page.

FIG. 11 is a screenshot of an exemplary Laws, Regulations, Compliance and Liabilities page.

FIG. 12 is a screenshot of an exemplary Physician Forum page.

FIG. 13 is a screenshot of an exemplary Upcoming Events page for both the physician and patient portal portions of the website.

FIG. 14 is a screenshot of an exemplary Blogs, News, and Updates page for both the physician and patient portal portions of the website.

FIG. 15 is an exemplary screenshot of a Log In for gaining access to the back end, paid for portion of the website.

FIG. 16A is an example data flow of the landing page for the website that directs physicians to our physician web portal and patients to our patient web portal.

FIG. 16B is an example of a flow chart of the physician back end of the present invention.

FIG. 16C is an example of a flow chart of the patient back end of the present invention.

FIG. 17 is a screen shot of an exemplary Home Page.

FIG. 18A is a screen shot of an exemplary Physician Portal landing page.

FIG. 18B is a screenshot of an exemplary Patient Portal landing page.

FIG. 19 is a screen shot of an exemplary About Us page.

FIG. 20A is a screenshot of an exemplary Physician Member Sign Up and Registration Page.

FIG. 20B is a screenshot of an exemplary Patient Member Sign Up and Registration Page.

FIG. 21 is a screenshot of an exemplary Patient-Member Site Directory page for the patient portal portion of the website.

FIG. 22 is a screenshot of an exemplary Peer Reviewed and Anecdotal Medical Cannabis Research page for the patient portal portion of the website.

FIG. 23A is a screenshot of an exemplary Medical Marijuana Treatment Center Product Knowledge page for the physician portal portion of the website

FIG. 23B is a screenshot of an exemplary Medical Marijuana Treatment Center Product Knowledge page for the patient portal portion of the website

FIG. 24, is a screenshot of an exemplary Our Patient Initiatives page for the physician portal portion of the website.

FIG. 25 is a screenshot of an exemplary Our Physician-Members page for the patient portal portion of the website.

FIG. 26A is a screenshot of an exemplary Laws, Regulations, Compliance and Liabilities page for the physician portal portion of the website.

FIG. 26B is a screenshot of an exemplary Laws, Regulations, Compliance and Liabilities page for the patient portal portion of the website.

FIG. 27A is a screenshot of an exemplary Physician Forum page for the physician portal portion of the website.

FIG. 27B is a screenshot of an exemplary Patient Forum page for the patient portal portion of the website.

FIG. 28 is an exemplary screenshot of a Log In landing page for the physician and patient portions of the website.

FIG. 29 is an exemplary screenshot of our the Product Page of our Store for the physician and patient portions of the website.

### DETAILED DESCRIPTION OF THE INVENTION

In the following description, for purposes of explanation and not limitation, specific details are set forth, such as particular networks, communication systems, computers, terminals, devices, components, techniques, storage devices, data and network protocols, software products and systems, operating systems, development interfaces, hardware, etc. in order to provide a thorough understanding of the present invention. However, it will be apparent to one skilled in the art that the present invention can be practiced in other embodiments that depart from these specific details. Detailed descriptions of well-known networks, computers, digital devices, storage devices, components, techniques, data and network protocols, software products and systems, development interfaces, operating systems, and hardware are omitted so as not to obscure the description of the present invention. All use of the word “example” are intended to describe non-limiting examples of the invention.

The invention will now be explained with reference to the attached non-limiting Figures. The operations described in the Figures and herein can be implemented as executable code stored on a computer or machine readable non-transitory tangible storage medium (e.g., floppy disk, hard disk, ROM, EEPROM, nonvolatile RAM, CD-ROM, etc.) that are completed based on execution of the code by a processor circuit implemented using one or more integrated circuits; the operations described herein also can be implemented as executable logic that is encoded in one or more non-transitory tangible media for execution (e.g., programmable logic arrays or devices, field programmable gate arrays, programmable array logic, application specific integrated circuits, etc.).

To facilitate an understanding of the principles and features of the various embodiments of the present invention, various illustrative embodiments are explained below. Although example embodiments of the present invention are explained in detail, it is to be understood that other embodiments are contemplated. Accordingly, it is not intended that the present invention is limited in its scope to the details of construction and arrangement of components set forth in the following description or examples. The present invention is capable of other embodiments and of being practiced or carried out in various ways.

As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Also, in describing the example embodiments, terminology will be resorted to for the sake of clarity. It is intended that each term contemplates its broadest meaning as understood by those skilled in the art and includes all technical equivalents that operate in a similar manner to accomplish a similar purpose.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified. Such other components or steps not described herein can include, but are not limited to, for example, similar components or steps that are developed after development of the disclosed technology.

As illustrated, lines or arrows between elements can denote communications between the different elements. These communications can take any form known by those of skill in the art, including digital, telephonic, or paper. The communications can be through a WAN, LAN, analog phone line, etc. The information communicated can be in any format appropriate for the transmission medium.

“Data storage” can be non-transitory tangible memory, such as any one or a combination of a hard drive, random access memory, flash memory, read-only memory and a memory cache, among other possibilities. The data storage can include a database, implemented as relational database tables or structured XML documents or any other format. Such a database can be used to store the information gathered from transaction records and Thing Records. Non-volatile memory is preferred.

“Processor” can refer to a single data processor on a single computing device or a collection of data processors. The collection of data processors can reside on a single computing device or be spread across multiple computing devices. The processor can execute computer program code stored in the data storage or a memory. In one example, the processor can execute computer program code representative of functionalities of various components of the system.

While certain implementations of the disclosed technology have been described in connection with what is presently considered to be the most practical and various implementations, it is to be understood that the disclosed technology is not to be limited to the disclosed implementations, but, on the contrary, is intended to cover various modifications and equivalent arrangements included within the scope of the appended claims. Although specific terms are employed herein, they are used in a generic and descriptive sense only and not for purposes of limitation.

Certain implementations of the disclosed technology are described above with reference to block and flow diagrams of systems and methods and/or computer program products according to example implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams do not have to be performed in the order presented or if at all, according to some implementations of the disclosed technology.

These computer program instructions can also be stored in a non-transient computer-readable memory that can direct a computer or other programmable data processing apparatus to function in a particular manner, such that the instructions stored in the computer-readable memory produce an article of manufacture including instruction means that implement one or more functions specified in the flow diagram block or blocks.

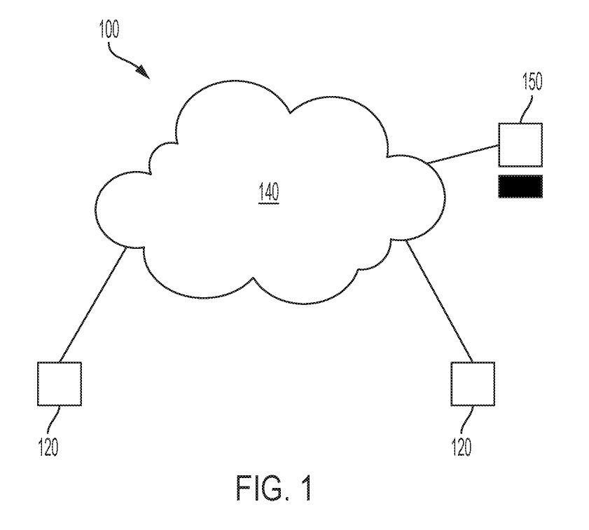

FIG. 1 describes an example of a system 100. The system 1 comprises user interface devices 120, a server 150, all interconnected via a communication network 140. All interconnections can be direct, indirect, wireless and/or wired as desired.

The network 140 can be any desired network including the internet or telephone network. Various networks 140 can be implemented in accordance with embodiments of the invention, including a wired or wireless local area network (LAN) and a wide area network (WAN), wireless personal area network (PAN) and other types of networks that comprise or are connected to the Internet. When used in a LAN networking environment, computers can be connected to the LAN through a network interface or adapter. When used in a WAN networking environment, computers typically include a modem, router, switch, or other communication mechanism. Modems can be internal or external, and can be connected to the system bus via the user-input interface, or other appropriate mechanism. Computers can be connected over the Internet, an Intranet, Extranet, Ethernet, or any other system that provides communications, such as by the network. Some suitable communications protocols can include TCP/IP, UDP, OSI, Ethernet, WAP, IEEE 802.11, Bluetooth, Zigbee, IrDa, WebRTC, or any other desired protocol. Furthermore, components of the system can communicate through a combination of wired or wireless paths, including the telephone networks.

The system 100 can be accessed via any user interface device 120 that is capable of connecting to the server 150 via the network 140. A plurality of user interface devices 120 can be connected to the server 150. An example user interface device 120 contains a web browser and display. This includes user interface devices 120 such as internet connected televisions and projectors, tablets, iPads, Mac OS computers, Windows computers, e-readers, and mobile user devices such as the smartphones, iPhone, Android, and Windows Phone, and other communication devices. The user interface device 120 is a smartphone. The smartphone 120 can be in any form, such as a hand held device, wristband, or part of another device, such as vehicle.

The computer processing unit (CPU) of the user interface device 120 can be implemented as a conventional microprocessor, application specific integrated circuit (ASIC), digital signal processor (DSP), programmable gate array (PGA), or the like. The CPU executes the instructions that are stored in order to process data. The set of instructions can include various instructions that perform a particular task or tasks, such as those shown in the appended flowchart. Such a set of instructions for performing a particular task can be characterized as a program, software program, software, engine, module, component, mechanism, or tool. The non-transitory memory can include random access memory (RAM), ready-only memory (ROM), programmable memory, flash memory, and the like. The memory, include application programs, OS, application data etc.

The server 150 described herein can include one or more computer systems directly connected to one another and/or connected over the network. Each computer system can include a processor, non-transitory memory, user input and user output mechanisms, a network interface, and executable program code (software) comprising computer executable instructions stored in non-transitory tangible memory that executes to control the operation of the server 150. Similarly, the processors functional components formed of one or more modules of program code executing on one or more computers. Various commercially available computer systems and operating system software can be used to implement the hardware and software. The components of each server can be co-located or distributed. In addition, all or portions of the same software and/or hardware can be used to implement two or more of the functional servers (or processors) shown. The server 150 can run any desired operating system, such as Windows, Mac OS X, Solaris or any other server based operating systems. Other embodiments can include different functional components. In addition, the present invention is not limited to a particular environment or server 150 configuration. Preferably, the server 150 is a cloud based computer system. If desired for the particular application, the server 150 or portions of the server 150 can be incorporated within one or more of the other devices of the system 100, including but not limited to a user interface device 120.

The server 150 includes at least one web server and the query processing unit. The web server receives the user query and sends the user query to the query processing unit. The query processing unit processes the user query and responds back to the user interface device 120 via the web server. The query processing unit fetches data from the database server if additional information is needed for processing the user query. The database is stored in a non-transitory tangible memory, and preferably a non-volatile memory. The term “database” includes a single database and a plurality of separate databases. The server 150 can comprise the non-volatile memory or the server 150 can be in communication with the non-volatile memory storing the database. The database can be stored at different locations.

Software program modules and data stored in the non-transitory memory the server 150 and/or non-volatile memory of the user interface device 120 can be arranged in logical collections of related information on a plurality of computer systems having associated non-volatile memories. The software and data can be stored using any data structures known in the art including files, arrays, linked lists, relational database tables and the like. The server 150 and mobile user device 120 can be programmed to perform the processes described herein.

Implementations of the disclosed technology can provide for a computer program product, comprising a computer-usable medium having a computer-readable program code or program instructions embodied therein, said computer-readable program code adapted to be executed to implement one or more functions specified in the flow diagram block or blocks. The computer program instructions can also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

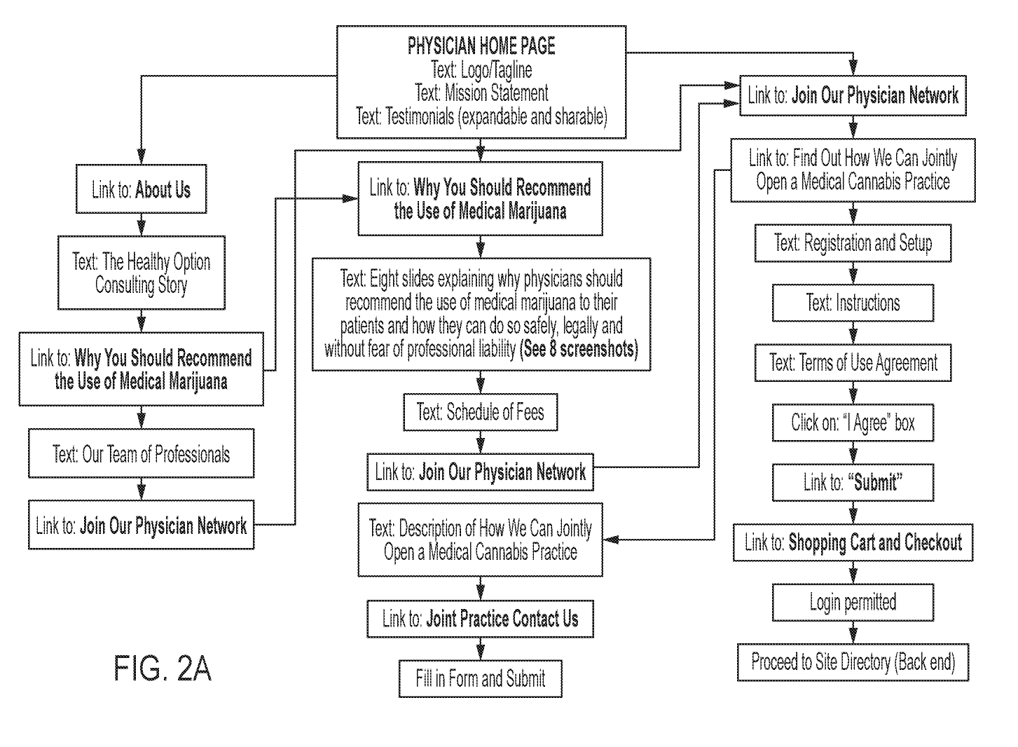

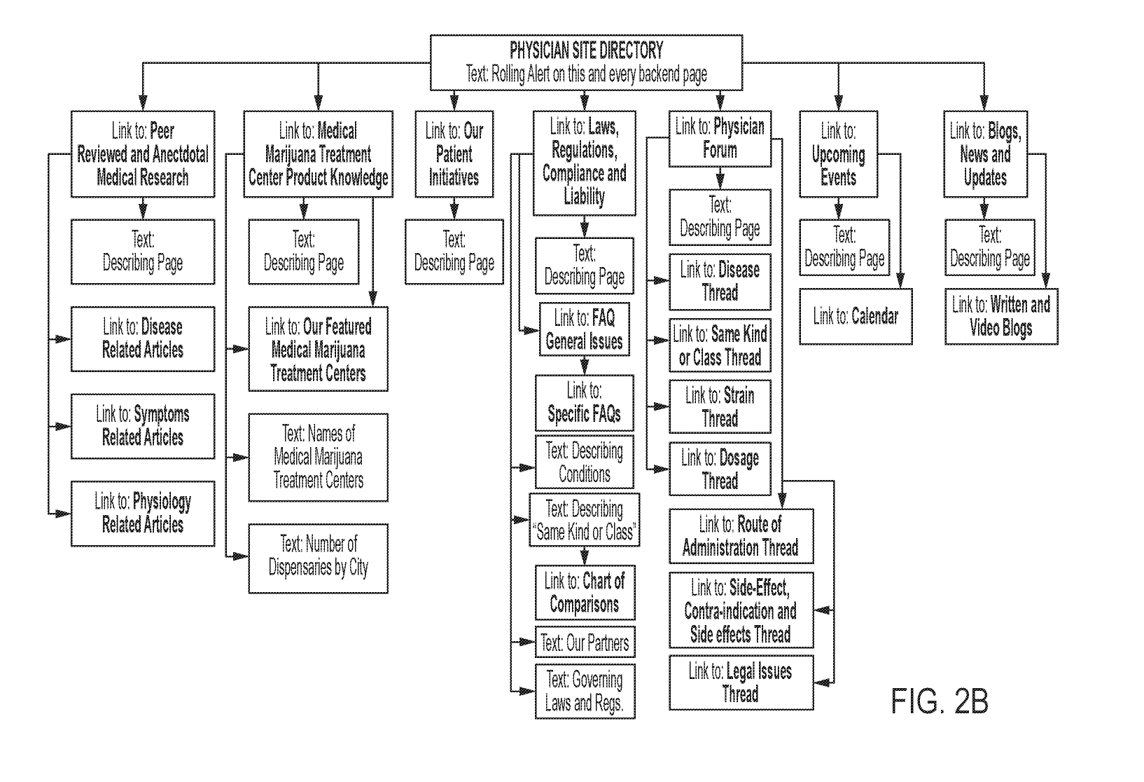

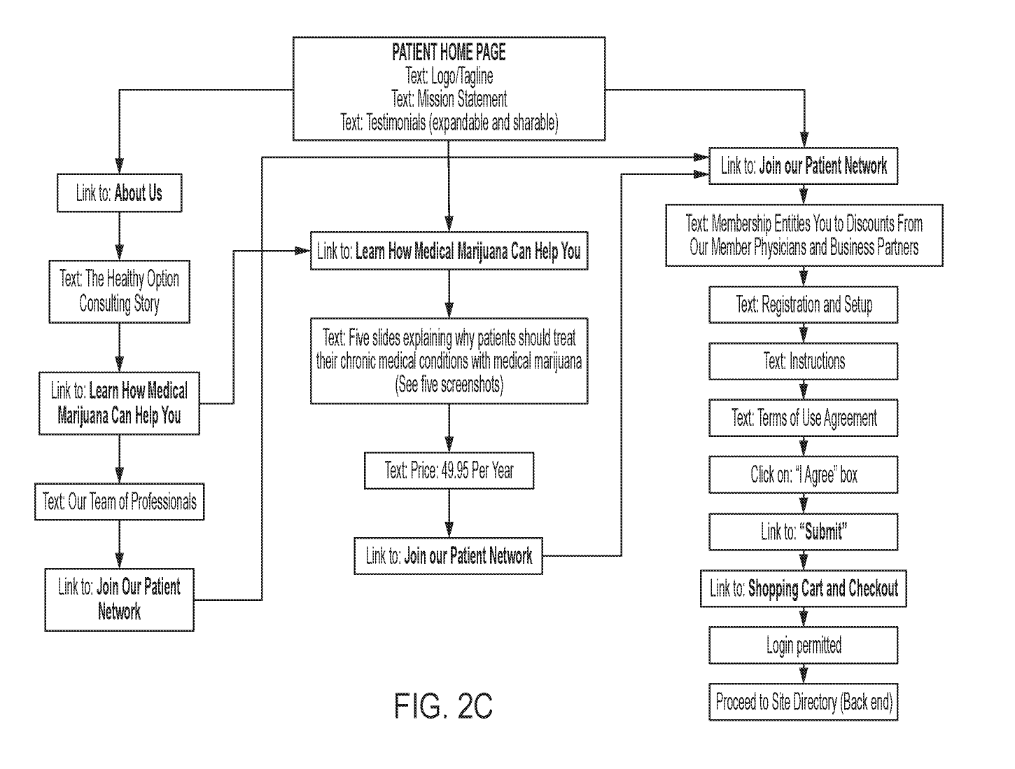

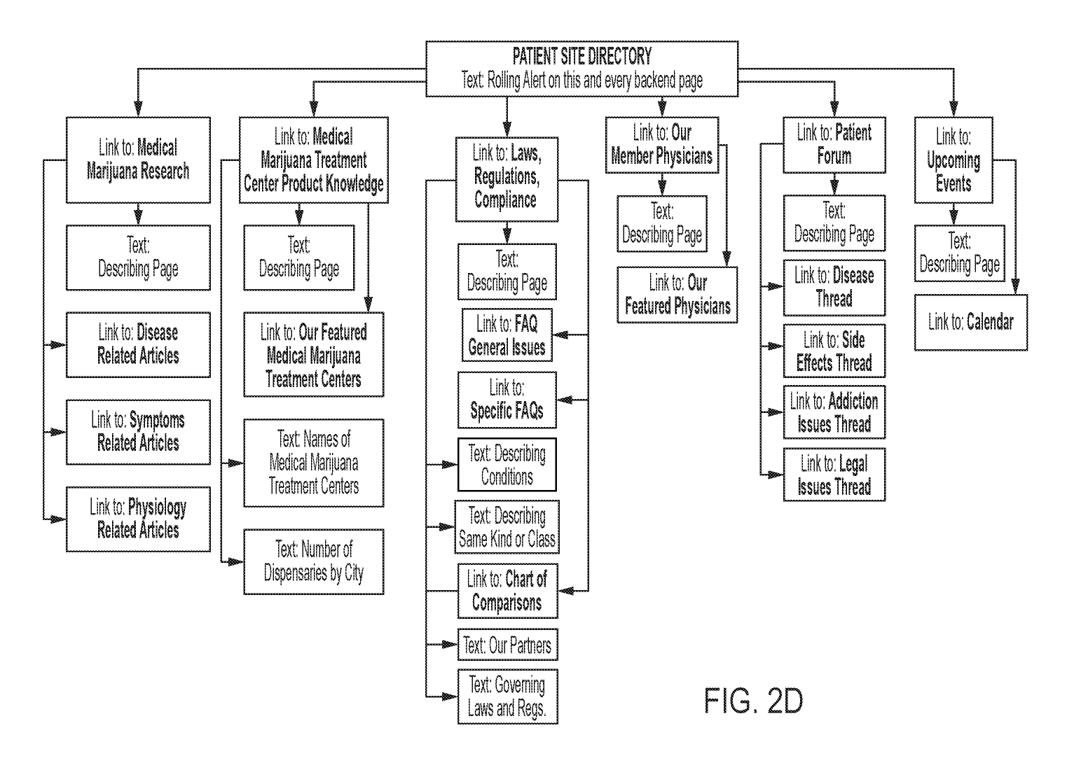

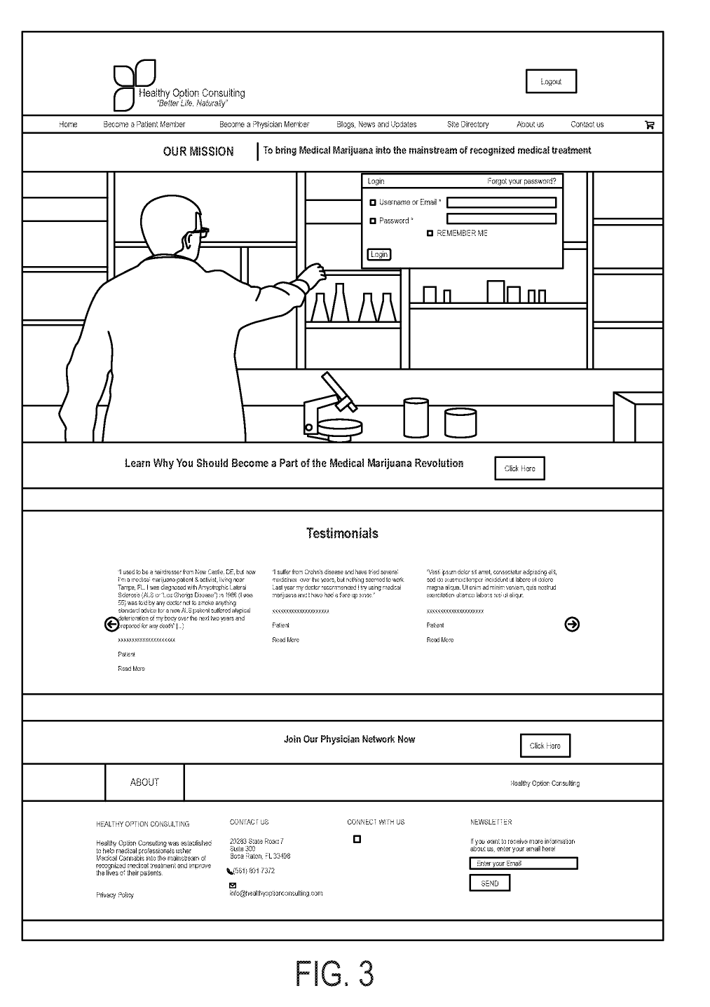

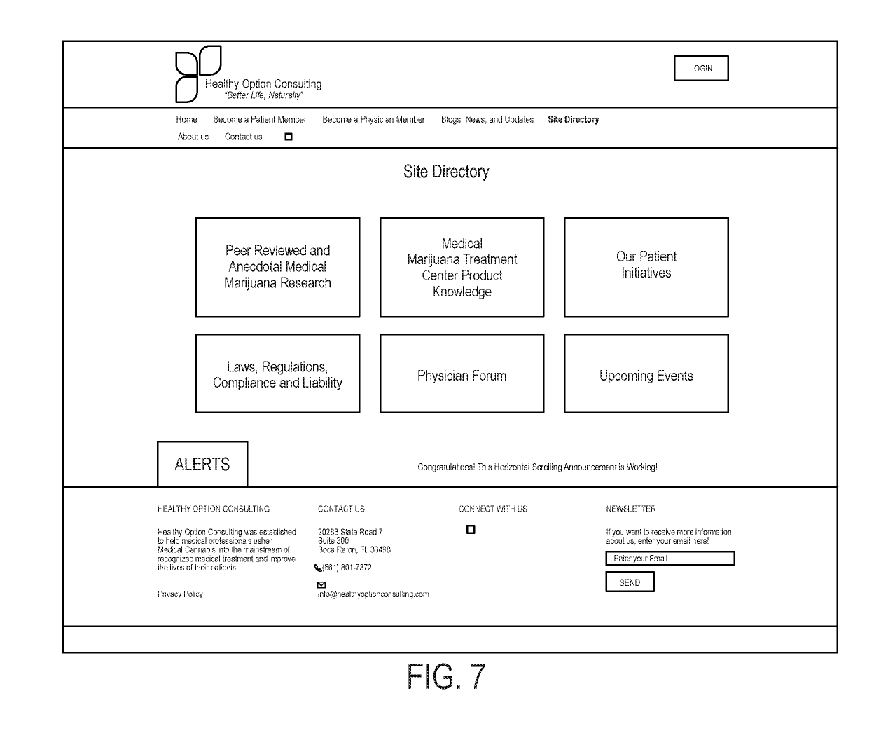

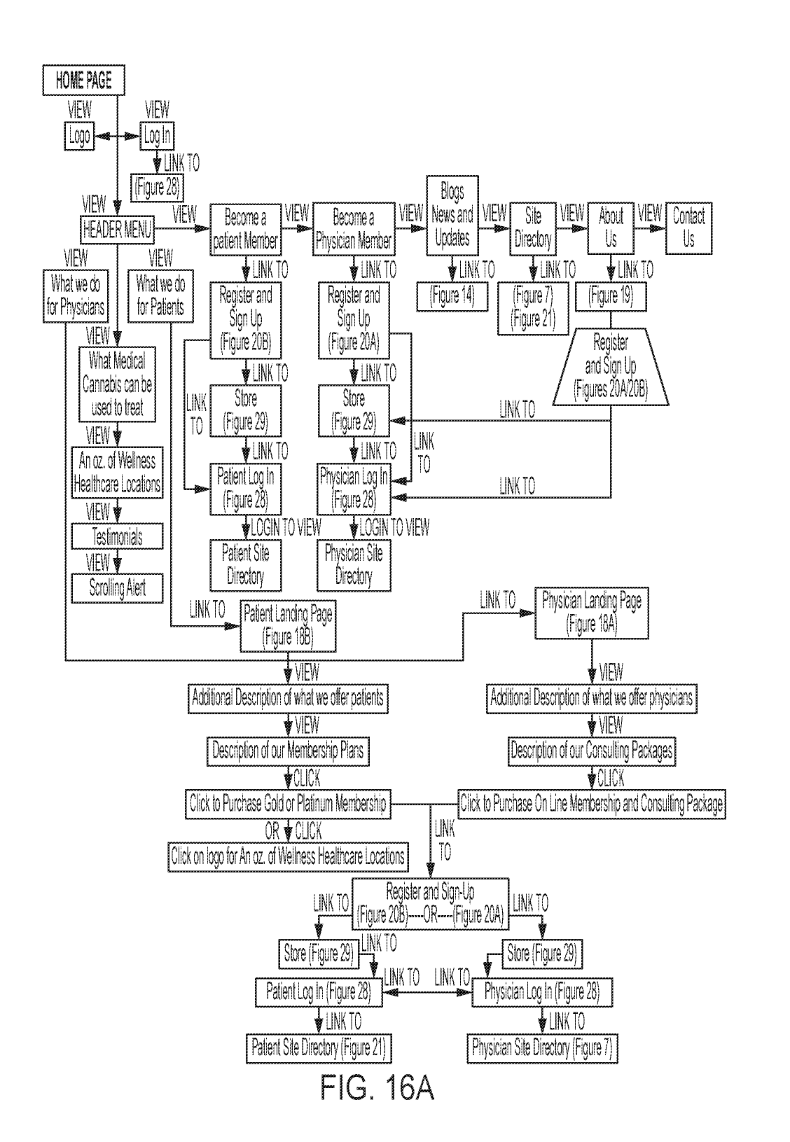

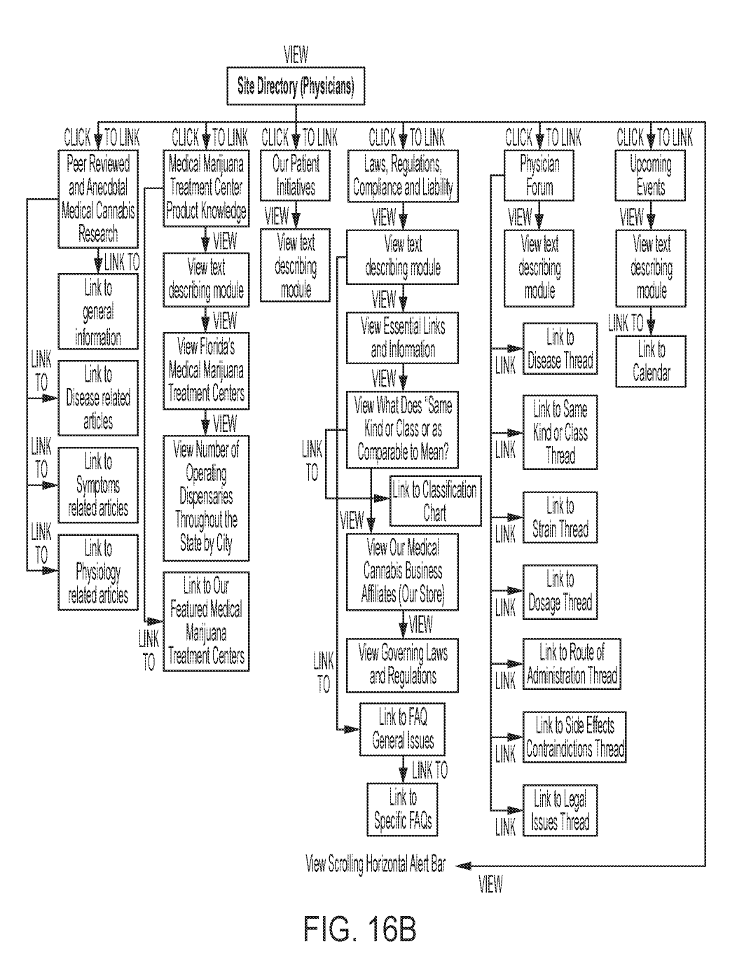

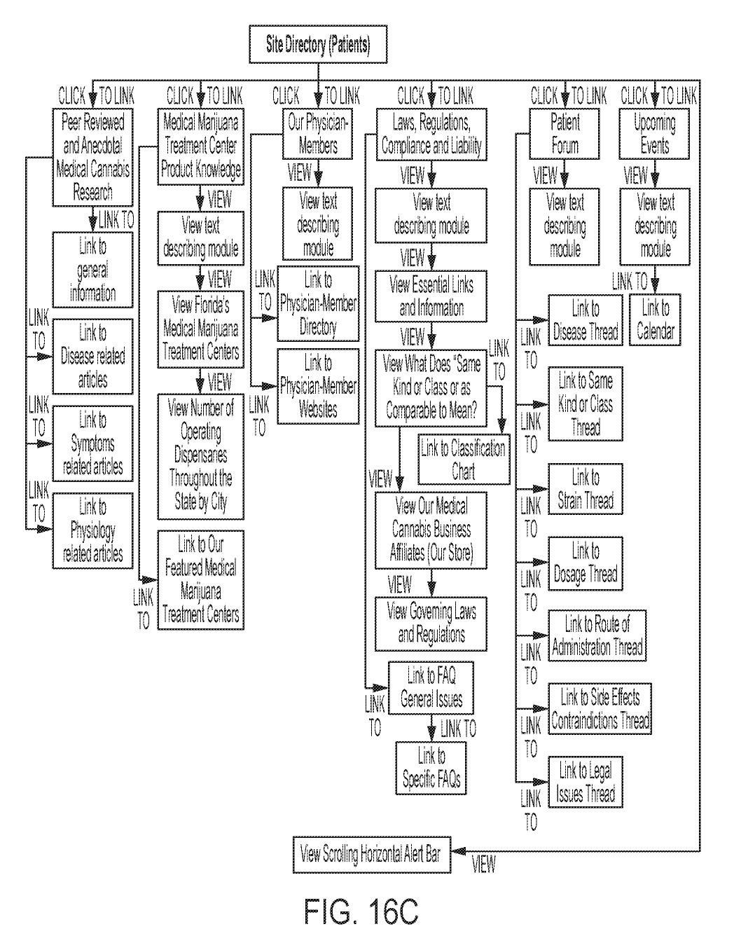

FIGS. 2A-2D illustrates a flow chart of a first exemplary system and method, which will be described in reference to the attached FIGS. 3-15 of exemplary screenshots. FIGS. 16A-16C illustrate a flow chart a second exemplary system and method, which will be described in reference to the attached FIGS. 7, 8, 13, 14, and 17-29 of exemplary screenshots. The following definitions are used herein:

Use of the term “system” refers to the methods and systems disclosed herein.

The term “cannabis” refers to an annual, dioecious, flowing plant in the family of

Cannabaceae. Cannabis can also be referred to as marijuana, bhang, hashish, hemp, pot, grass and reefer. Cannabis includes psychoactive compounds, including but not limited to Delta-9 tetrahydrocannabinol (THC), non-psychoactive compounds, including but not limited to cannibidiol (CBD), and other cannabinoids, terpenoids or flavonoids. All cannabinoids, terpenoids, flavonoids and any other compounds naturally found in cannabis are included in the present invention, with THC and CBD being the most prevalent. Medical cannabis can be administered to a patient in need thereof by any method, including but not limited to sublingual, oral, topical, inhaled (combusted or vaporized), or rectal, and in any form, such as natural flower, oil, tincture, ointment, salve or composition.

The term “medical practitioner” includes doctors, physicians, and any other medical professional having the ability to recommend the use of medical cannabis to patients.

The term “patient information” can include for example typical information included in medical records, such as age, sex, medical history, symptoms, treatment, and present care. Preferred information includes any information relating to medical cannabis, such as dosage, administration instructions, reasons for treatment, symptoms, success of treatment, complications, side effects, or any other information. The patient information can also include information on prospective patients.

The term “patient” includes patients currently using medical cannabis and patients that may be in need of medical cannabis. The term may also refer to prospective patients.

The term “patient database” refers to a patient database that can be stored in the memory on the server that relates to patient information.

The term “legal database” refers to a legal database that can be stored in the memory on the server containing information relating to any of lawyers, compliance experts, and cannabis related laws and rules.

The term “medical practitioner database” refers to a medical practitioner database that can be stored in the memory on the server containing information relating to medical practitioners.

The term “medical cannabis database” relates to a medical cannabis database that can be stored in the memory on the server, containing information relating to medical cannabis including but not limited to strains, constituent compounds, dosing and treatment protocols, effects, drug interactions, side effects, and compositions.

The term “cannabis supplier database” relates to a cannabis supplier database that can be stored in the memory on the server, containing information relating to medical cannabis growers, processors, and dispensaries.

The term “cannabis banking database” relates to a cannabis banking database that can be stored in the memory on the server, containing information relating to banks, lenders and other financial institutions that will conduct business with the cannabis industry.

Each of the databases described herein, software, and other information used by the system can be store in the memory, and/or on a memory remote to the server, with the proviso that the processor is in communication with the memory on the server or remote memory.

### EXAMPLE 1

### The Screenshots Shown FIGS. 3-15 Represent A Working Website

Medical practitioners and others viewing the homepage shown in FIG. 3 have the opportunity to view testimonials offered by other healthcare providers or their patients who have benefited from the use of medical cannabis so that they may learn about its therapeutic effects, as well as a scrolling alert bar displaying additional messaging.

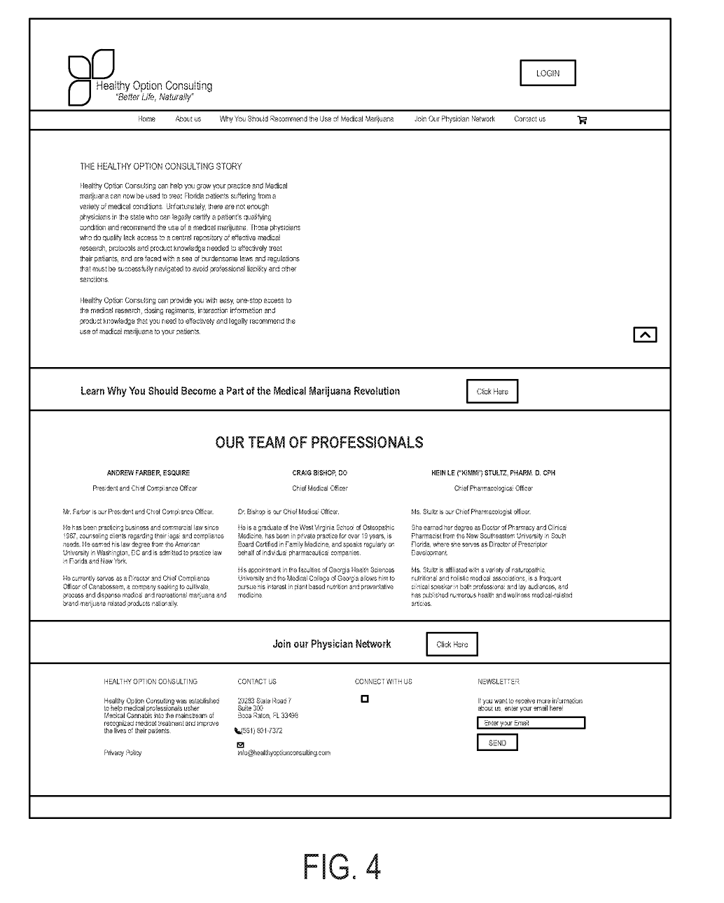

For medical practitioners to learn why they should recommend the use of medical cannabis and sign up on the system as Physician-Members, they can click on the header tab “Why You Should Recommend the Use of Medical Marijuana” at the top of the home page (FIG. 3), click on the button “Learn Why You Should Become a Part of the Medical Marijuana Revolution” at the bottom of the home page, or click on the button “Learn Why You Should Become a Part of the Medical Cannabis Revolution” on the “About Us” page (FIG. 4). By doing so, they are directed to a new page that describes the following features that the system offers to medical practitioners.

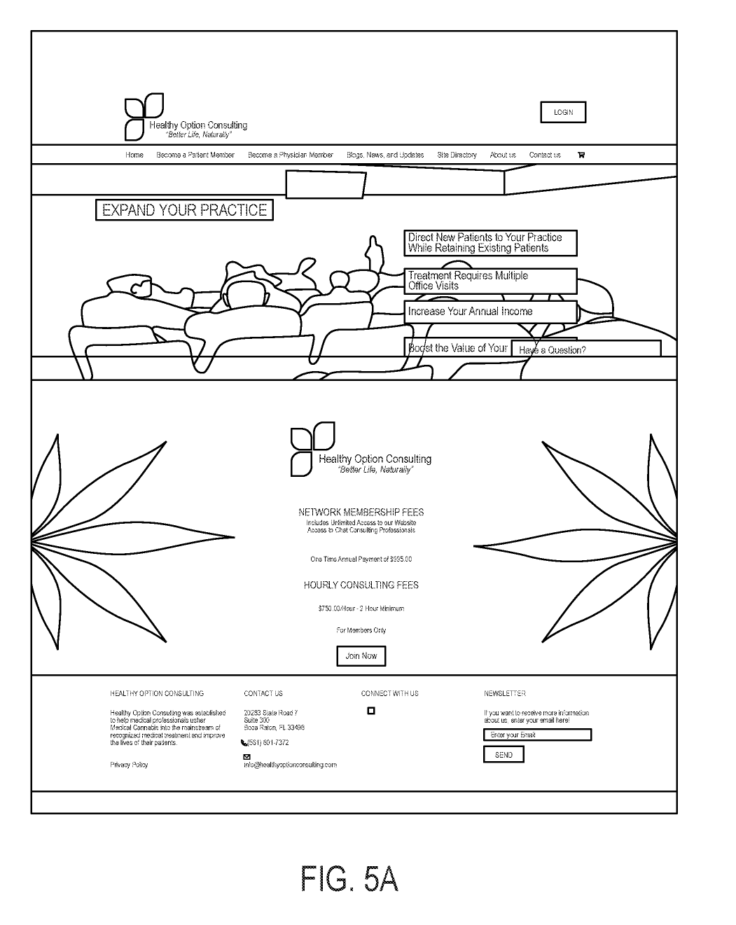

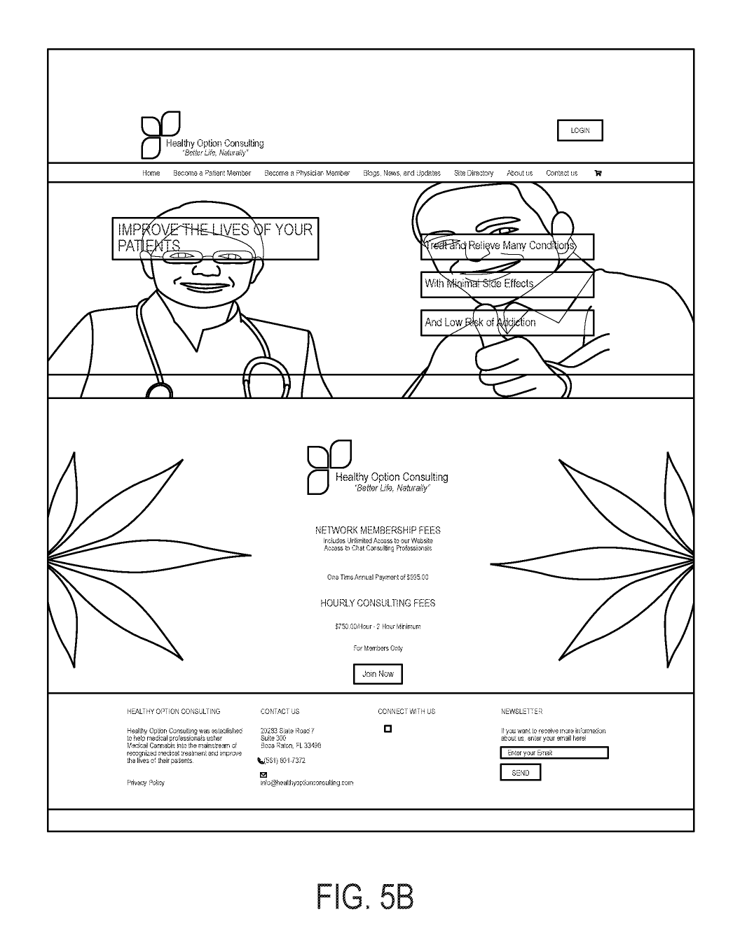

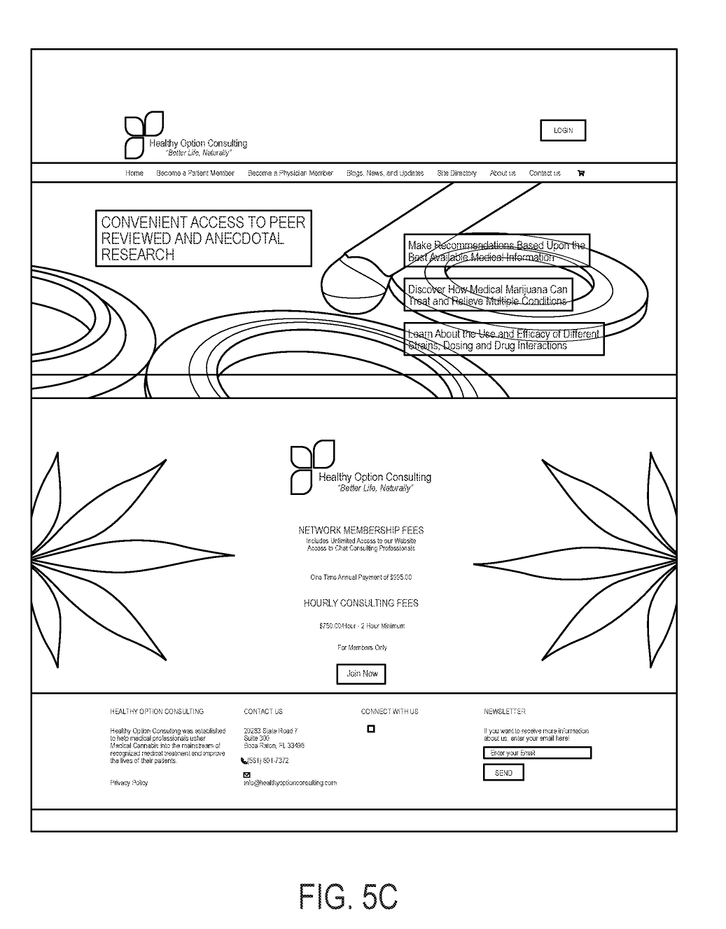

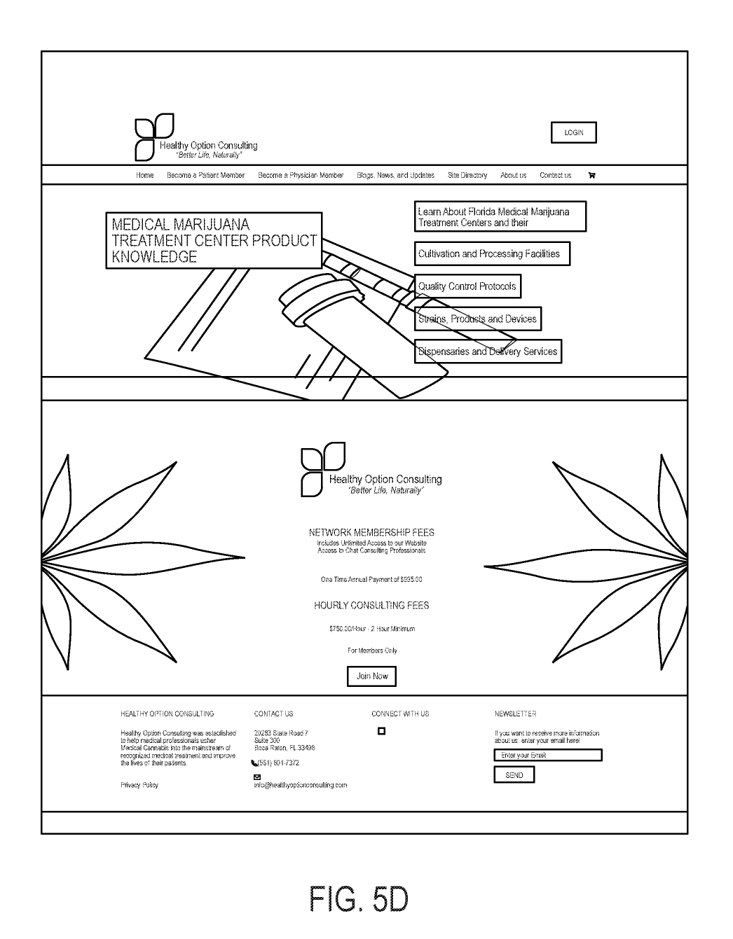

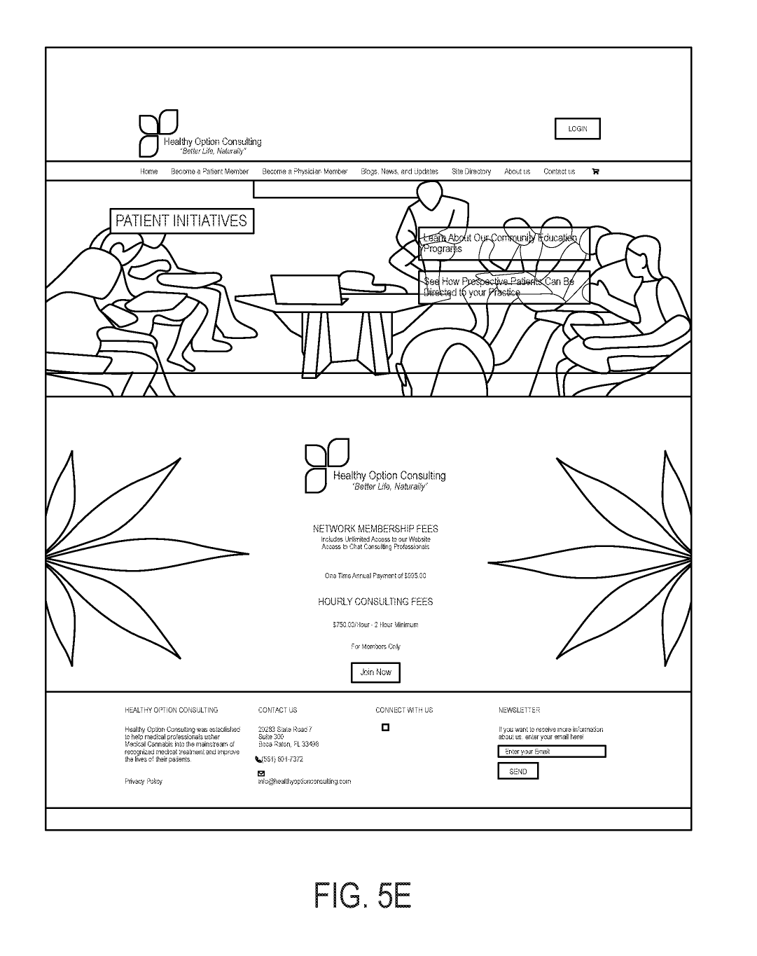

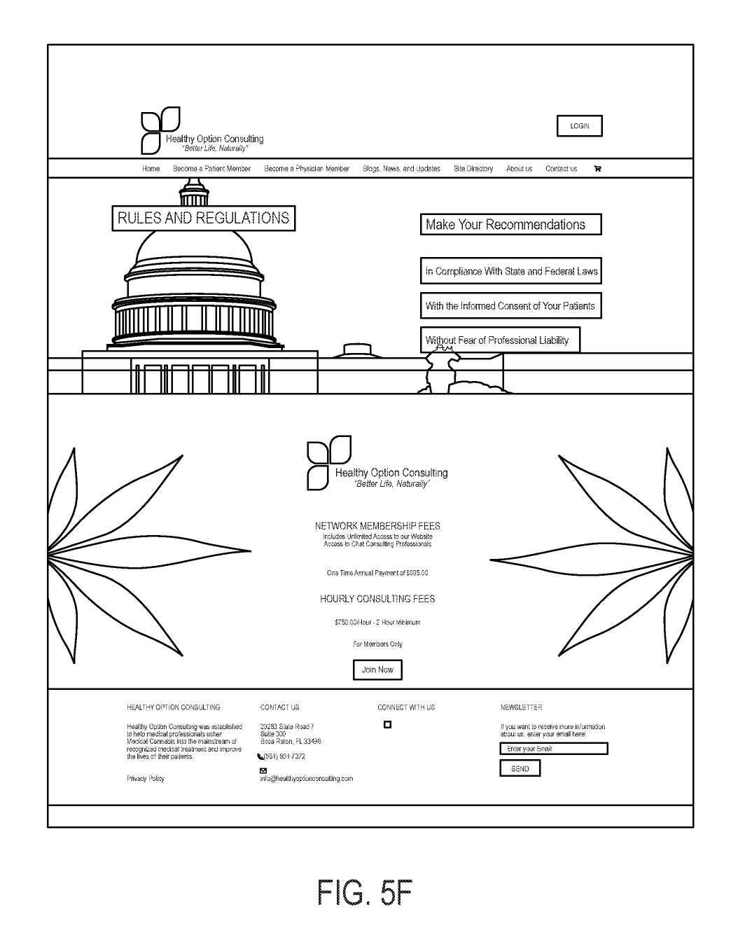

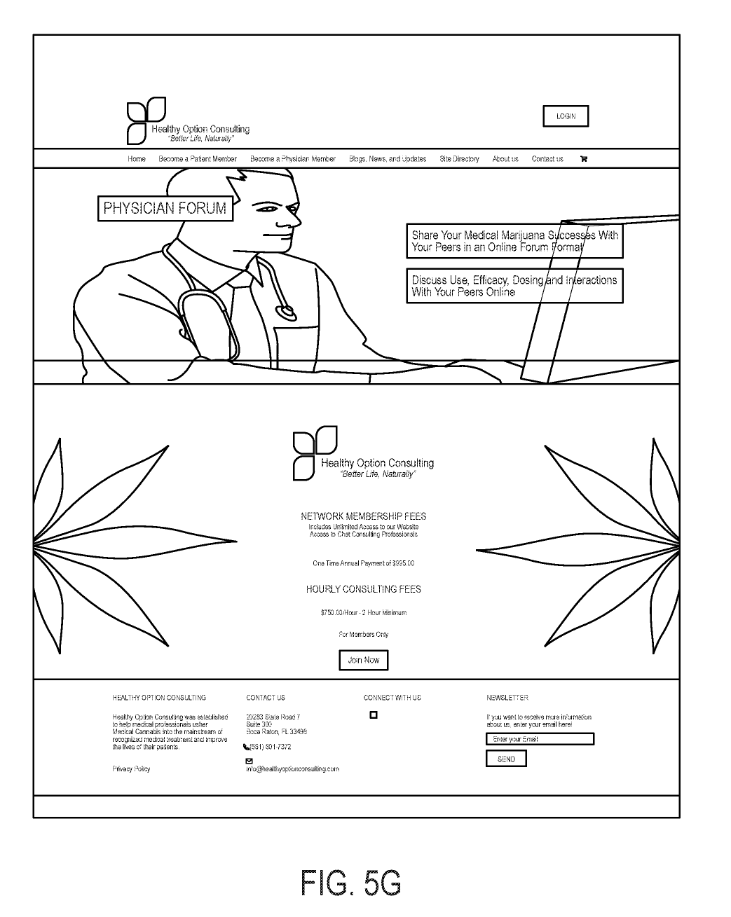

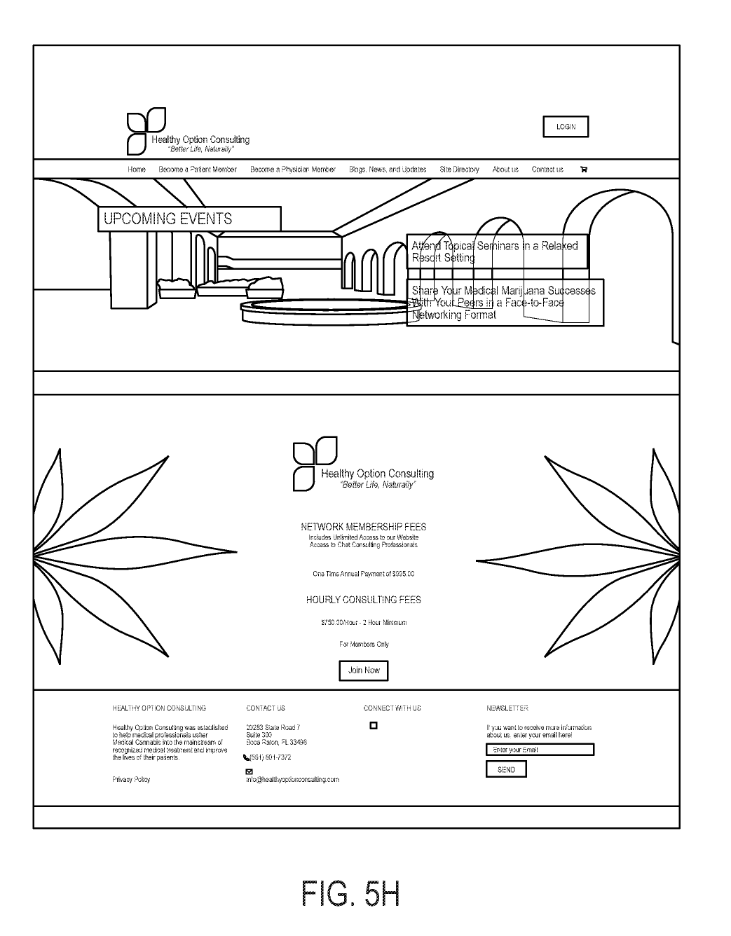

Expand Your Practice (FIG. 5A): Direct new patients to the your practice while retaining existing patients; Treatment requires multiple office visits; Increase your income and the valuation of the your practice.

Improve the Lives of Your Patients (FIG. 5B): Treat and relieve many conditions with minimal side effects and low risk of addiction.

Convenient Access to Peer Reviewed and Anecdotal Research (FIG. 5C): Make recommendations based on the best available medical information; Discover how medical cannabis can treat and relieve multiple conditions; Learn about the use and efficacy of different strains; Dosing and titration protocols; Drug interactions and contra-indications.

Medical Marijuana Treatment Center Product Knowledge (FIG. 5D): Learn about Florida's Medical Marijuana Treatment Centers and their cultivation and processing facilities, quality control protocols, strains, products, delivery devices, dispensaries and delivery services.

Patient Initiatives (FIG. 5E): Learn about community education and outreach programs. See how prospective patients can be directed to your practice.

Rules and Regulations (FIG. 5F): Make your recommendations in compliance with federal and state law, with the informed consent of your patients, without fear of professional liability.

Physician Forum (FIG. 5G): Users can share their medical cannabis successes with their peers in an online forum format and discuss use, efficacy, dosing and interaction issues with their peers online.

Upcoming Events (FIG. 5H): Attend topical seminars in a relaxed online setting. Users can conduct continuing education credits and share their medical cannabis successes with peers in a face-to-face networking format.

These pages also contains a description of an exemplary system means of website access as well as our pricing.

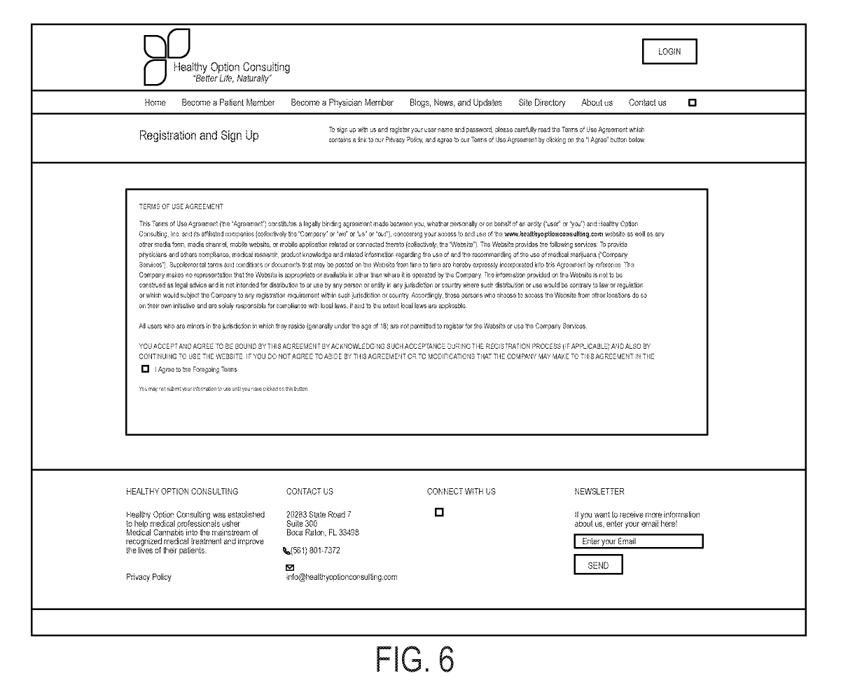

Sign Up: To sign up with the system and become a Physician-Member, the user can click on the Join our Physician Network link on the website's common header menu or the Join our Physician Network Now button at the bottom of the homepage (FIG. 3), the Join our Physician Network Button at the bottom of the “About Us” page (FIG. 4), or the Join Now button at the bottom of the “Why You Should Recommend the Use of Medical Cannabis” page (FIGS. 5A-5H). After agreeing to terms of use (FIG. 6), registering and making payment, Physician-Members gain access to the private, back end portion of the website where they can learn how to incorporate medical cannabis into their practices, learn how to make their medical cannabis recommendations to their patients safely, efficiently, profitably, legally and without fear of professional liability, and learn how the system can direct medical cannabis patients to their practices.

As an alternative means of signing up, or in addition thereto, the user can call or contact the Owner to provide medical cannabis practice development and training consulting services, or jointly develop, own, and operate a medical cannabis practice together with the Owner, whereby the Owner would be responsible for the operations and management of the medical cannabis practice using, among other things, the method and system comprising the invention.

Private access to the back end of the website teaches physicians how to incorporate medical cannabis into their practices, how to recommend medical cannabis to their patients safely, efficiently, profitably, legally and without fear of professional liability, and lets them know how we can drive medical cannabis patients to their practices.

Once Physician-Members gain access to the private, back end of our website by registering with and paying us, they are directed to our Physician Site Directory (FIG. 7), which displays seven links that Physician-Members may click on, that reference and link to the following modules which are more particularly described below: Peer Reviewed and Anecdotal Medical Cannabis Research; Florida's Medical Cannabis Treatment Centers and Their Products; Our Patient Initiatives; Laws, Regulations, Compliance and Liability; Physician Forum; Upcoming Events; and Blogs, News and Updates.

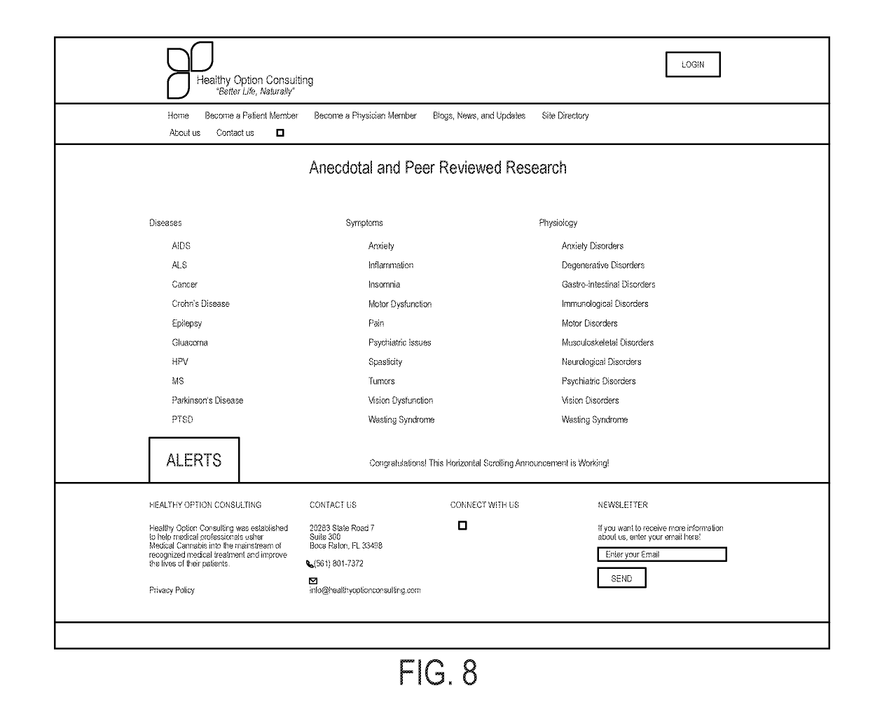

Peer Reviewed and Anecdotal Medical Cannabis Research (FIG. 8): This module provides Physician-Members with the clinical tools needed to safely conduct their medical cannabis practices in a fully compliant manner. It provides a library of medical cannabis research articles that are generally categorized by constitutionally permitted conditions for which medical cannabis may be used, symptoms and physiology. Each general category is then broken down into subcategories. As a user clicks on a subcategory, a page appears listing the research article(s) related to the subcategory. Physicians can then access our referenced medical articles by clicking on a pdf link to the extent one is available, or copying and pasting to an external site.

This module also contains links to medical cannabis practice protocols regarding dosing and titration, and contains additional content or links regarding, contraindications, side effects, and other clinical tools designed to help Physician-Members make their medical cannabis recommendations in connection with medical conditions that they may not regularly treat or be familiar with.

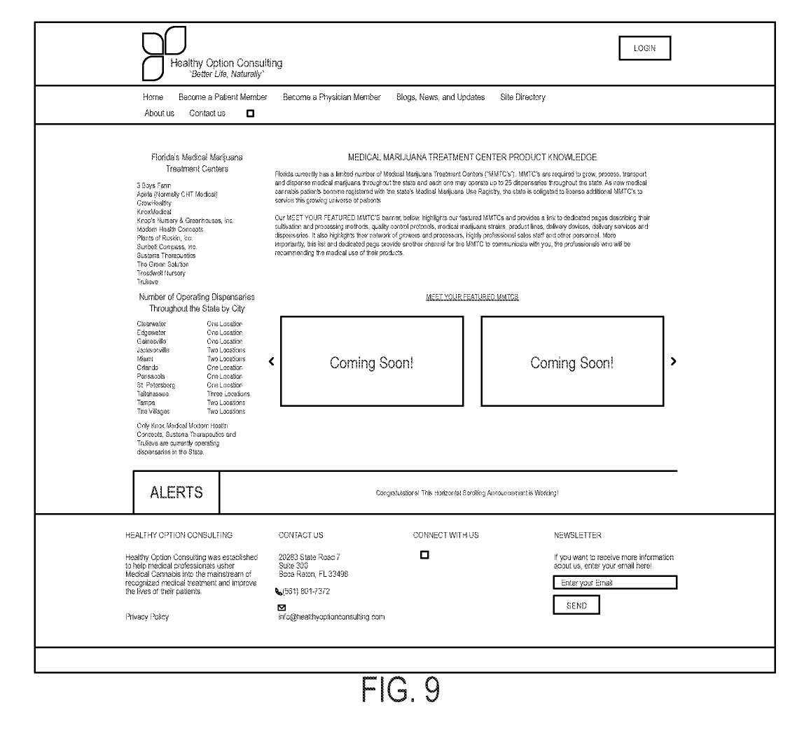

Florida's Medical Cannabis Treatment Centers and Products (FIG. 9): This module provides Physician-Members with information regarding Florida's Medical Marijuana Treatment Centers and their dispensaries. The left column on this page lists the Medical Marijuana Treatment Centers licensed to do business in Florida and the Florida cities within which their operating dispensaries are currently doing business.

This module also provides links to our featured Medical Marijuana Treatment Centers that are listed on the website for an annual fee. These links allow our Featured Medical Marijuana Treatment Centers to promote or educate users about their cultivation and processing methods, dispensaries, quality control protocols, product lines, and delivery services, and present their branding and other messaging. The links provides an additional means for them to communicate and do business with the medical professionals who recommend the use of their products and the patients who use them.

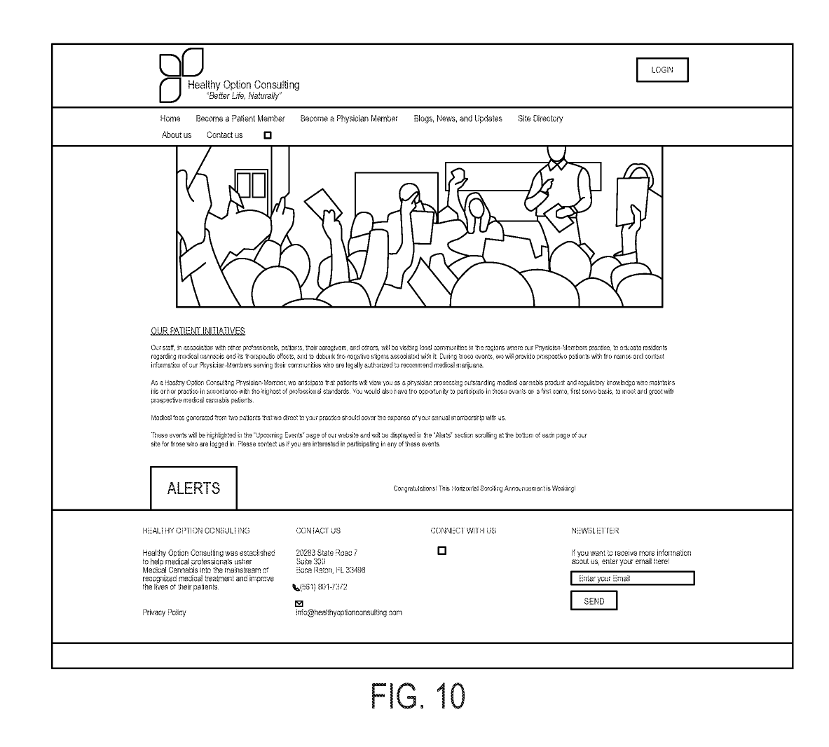

Our Patient Initiatives (FIG. 10): This module describes how the system can be used to undertake patient education and outreach community initiatives to teach the patient community at large about the medical benefits of medical cannabis, how to qualify for their Medical Marijuana ID Cards, and how they can find our Physician-Members and featured Dispensaries. It describes how: i) the Owner intends to hold regular speaking engagements featuring its staff and other speakers who can provide anecdotal examples of how medical cannabis has helped them or those that they know; ii) the system can be used by the Owner to detail the Patient-Member registration process and overall process for obtaining medical cannabis; and iii) the system can be used by the Owner to direct prospective patients to its Physician-Members and featured Dispensaries.

During the course of these initiatives, the system can also educate prospective patients about the Owner and let them know why they should become Patient-Members.

Patient Initiative events are reflected in the website's Upcoming Events calendar and are highlighted in the Alerts scrolling at the bottom of each page of the front end portion of the website.

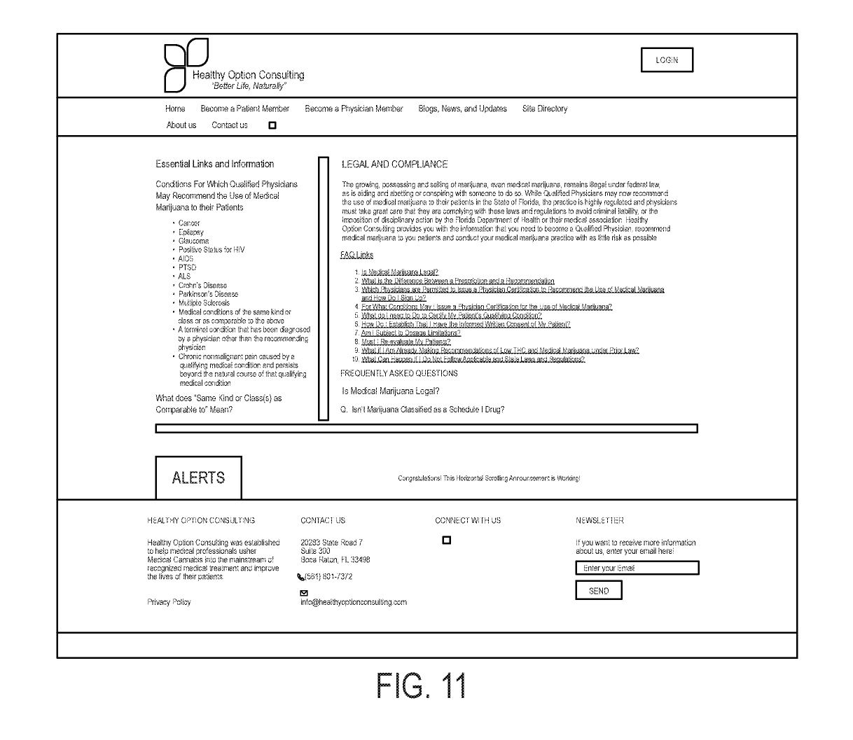

Laws, Regulations, Compliance and Liability (FIG. 11): This module contains the information needed by Physician-Members to enable them to operate their medical cannabis practices in compliance with Florida and federal law, and with low risk of professional liability. It also contains links to business affiliates who can offer Physician-Members discounted or full-priced medical cannabis and practice management related products and services that can be used in connection with their practices.

This module can be comprised of two columns. The left column contains Essential Links and Information, including a list of the conditions for which physicians may recommend the use of medical cannabis under Florida law and an analysis of what the term “same kind or class or as comparable to” means. This analysis can be accessed by clicking on a link to a proprietary Classification Chart, which can be used to assist physicians with their determination as to what medical conditions are of the same kind or class or otherwise comparable to those enumerated constitutional conditions for which medical cannabis may be used. The remainder of the left column contains a listing of business affiliates who offer their products and services for sale, as well as a link to the Florida laws and constitutional provision allowing the use of medical cannabis in Florida.

The right column contains information describing the compliance requirements imposed on physicians seeking to recommend the use of medical cannabis to their patients along with a brief analysis of these requirements.

This compliance related information is presented in the form of a series of frequently asked questions categorized into ten generalized topics. The issues raised in connection with each of these generalized topics are addressed by a group of specific related questions and answers which, when viewed as a whole and considered in conjunction with other information contained in the website, can enable the physician to make medical cannabis recommendations safely and with minimal legal and professional liability risk.

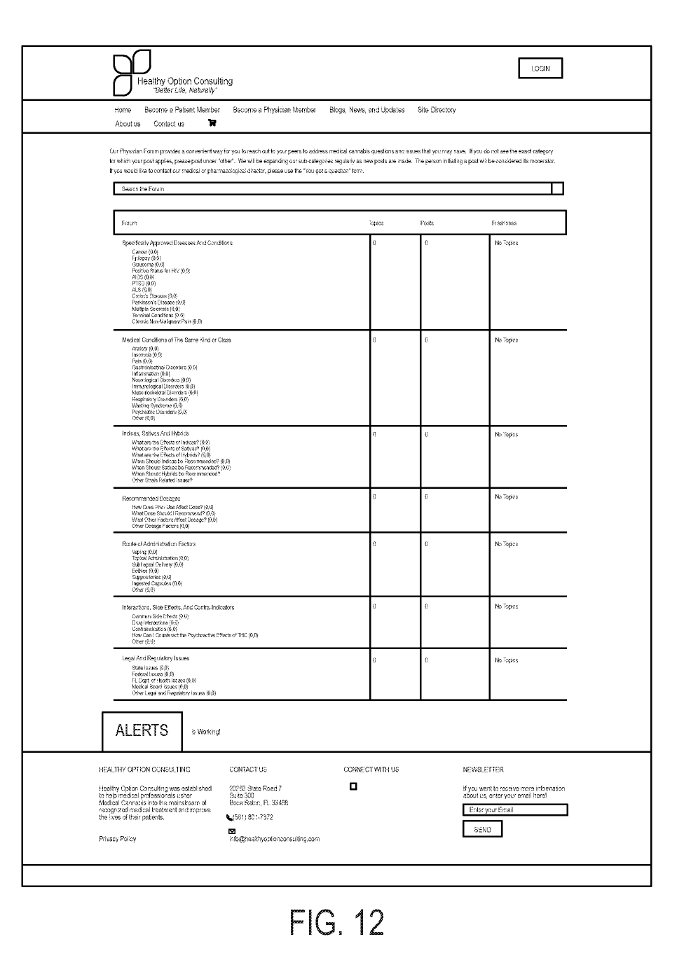

Physician Forum (FIG. 12): This module contains an interactive on-line forum designed to enable our Physician-Members to reach out to their peers and to the staff of the Owner to address medical cannabis related questions or concerns they may have, to share their case studies and clinical successes, and to provide the Physician-Member community with their practice related insights. The Physician Forum is composed of multiple discussion categories and subcategories which can be readily expanded should the need arise. The user initiating a discussion is considered the thread's moderator, and by posting a question, concern or issue, solicits responses from the system and its community of Physician-Members and other participants. A primary purpose of the Physician Forum is to lead to the overall improvement in the understanding of medical cannabis and its therapeutic effects by physicians, to help Physician-Members make their medical cannabis recommendations, and to aid in the establishment of new medical cannabis clinical protocols.

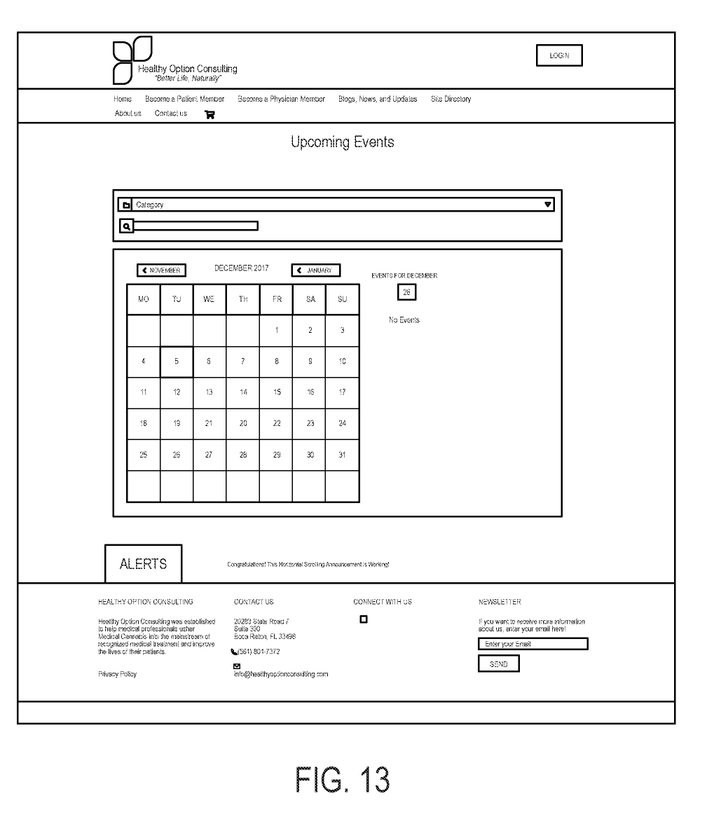

Upcoming Events (FIG. 13): This module contains a calendar of all future events that the Owner and system will be sponsoring. It contains a search bar that can enable the user to search for a particular type of event. Upcoming community speaking engagements as well as annual conferences can be reflected in the Upcoming Events calendar and can also be featured in the scrolling Alerts appearing at the bottom of each page of the back end of the website.

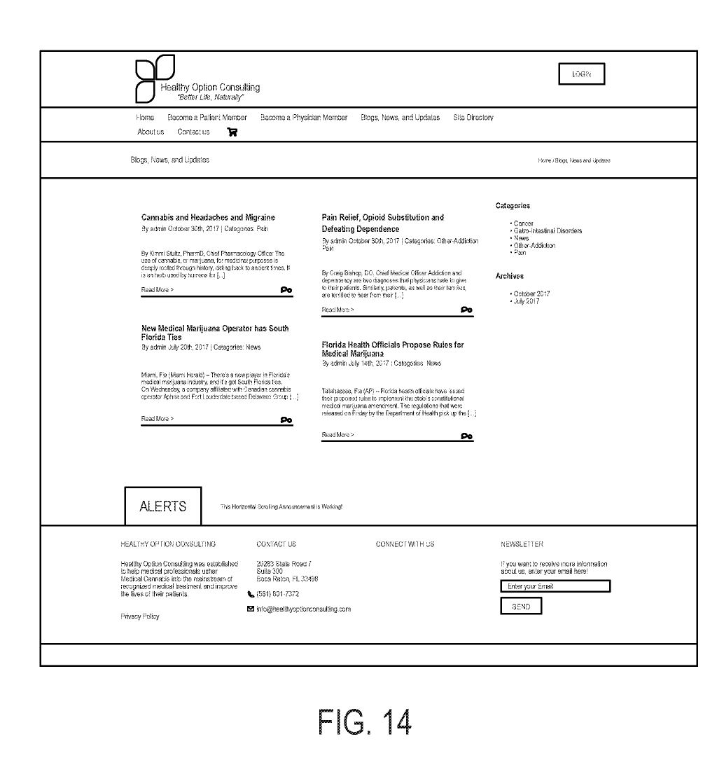

Blogs, News and Updates (FIG. 14): This module contains topical blogs authored by the medical, pharmacological, practice management, and compliance staff of the Owner, concerning their respective specialties and interests. Physician-Members may share these blog posts with others and leave comments for the author.

### Exemplary Website Map

Front End Function: Explains the system and why physicians looking to recommend the use of medical cannabis to their patients should join the network of physicians. It provides for physicians to join the system (Join our Network Button is at the bottom of each front end page appearing before the registration page).

Get to website by clicking on web address, https://www.healthyoptionconsulting.com.

View Home Page with header (Every page asks if you have a question so you can contact us), View our Mission Statement, View our Testimonials-Click on “Read More” to see balance of each testimonial, and View Alerts. Click to Read About the Management of the Owner. Click to Join our Network. Click to Learn Why You Should Recommend the Use of Medical Cannabis (pricing appears below slides).

View—Expand Your Practice: Direct New Patients to Your Practice While Retaining Existing Patients, Treatment Requires Multiple Office Visits, Increase Your Annual Income, and Boost the Value of Your Practice.

View—Convenient Access to Peer Reviewed and Anecdotal Research: Make Recommendations Based Upon the Best Available Medical Information, Discover How Medical Cannabis Can Treat and Relieve Multiple Conditions, Learn About the Use and Efficacy of Different Strains, Dosing, and Drug Interactions.

View—Medical Cannabis Treatment Center Product Knowledge: Learn About Florida's Medical Cannabis Treatment Centers and Their Cultivation and Processing Facilities. Quality Control Protocols. Strains, Products, and Devices. Dispensaries and Delivery Devices.

View—Patient Initiatives: Learn About our Community Education Programs, See How Prospective Patients Can Be Directed to Your Practice.

View—Rules and Regulations: Make Your Recommendations In Compliance With State and Federal Laws, With the Informed Consent of Your Patients, Without Fear of Professional Liability.

View—Physician Forum: Share Your Medical Cannabis Successes With Your Peers in an Online Format, Discuss Use, Efficacy, Dosing, and Interactions With Your Peers Online.

View—Upcoming Events: Attend Topical Seminars in a Relaxed Resort Setting, Share Your Medical Cannabis Successes With Your Peers in a Face-to-Face. Networking Format.

Click to Join Us: View Terms of Use.

Click to Agree to Terms of Use: Add your information to register. Make Payment and Join.

Click or Call to Contact Us to jointly develop, open and operate a medical cannabis practice with us.

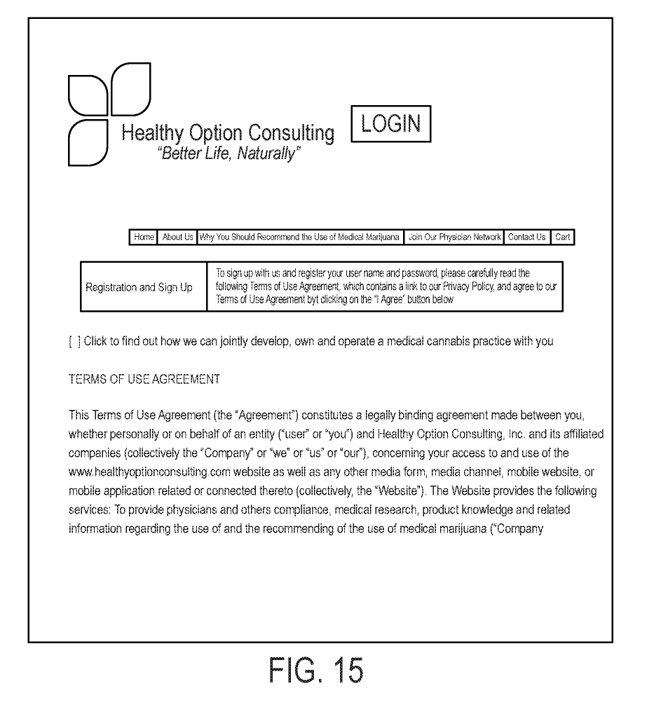

Click to Log In (FIG. 15).

Back End (Containing Paid For Content): Each page has a scrolling news Alert and “Have a Question?” link at the bottom and a header with a link for Blogs, News and Updates and the Site Directory.

View Site Directory (with six linked categorized buttons). Click on the appropriate button/link to see the desired module of the website that you would like to view.

Click on Peer Reviewed and Anecdotal Medical Cannabis Research: View columns of covered diseases. View columns of Symptoms. View columns of affected physiologies.

Click on each of the subcategories within the three columns and the user can be directed to a catalogue of medical cannabis research articles related to the linked category. If available, a copy of the article in pdf format can appear when the article link is clicked.

Click link on left column to reveal basic cannabis related information and medical cannabis dosage and titration guidelines.

Click link on left column to reveal drug interaction information.

Click link on left to reveal contraindication information.

Click link on left to reveal best practice guidelines.

Click on Medical Cannabis Treatment Centers and Their Products: View the Names of Medical Cannabis Treatment Centers on the left. View the number of statewide dispensaries by city on the left. View Medical Cannabis Treatment Center Product Knowledge on Right. View description of Meet Your Medical Cannabis Treatment Center feature on the right. View slides of featured Medical Cannabis Treatment Center.

Click on featured Medical Marijuana Treatment Center to view information about Florida's Medical Marijuana Treatment Centers, their dispensary locations, and information that our Featured Medical Marijuana Treatment Centers provide to us.

Click on Our Patient Initiatives—Describes how we intend to educate prospective patients regarding the health benefits of medical cannabis, the Owner, and our Physician-Members. Further describes how our initiatives can direct these prospective patients to the offices of our Physician-Members.

Click on Laws, Regulations, Compliance and Liability—Screen is comprised of narrow column on left and a wider column on the right. View Conditions for Which Medical Cannabis May be Recommended on the left. View analysis of “same kind or class or as comparable to” statutory provision.

Click on link to proprietary Classification Chart (on the left): View business partners of the Owner on the left. View governing laws (Florida Statutes and Constitution) on the left.

Click on link to provision of Florida Constitution relating to medical cannabis (on the left). View Introduction to Legal and Compliance section on the right. View Legal and Compliance tutorial which is set up as a series of Frequently Asked Questions (FAQs). View link of FAQ categories.

Click on link to FAQ master categories to be directed to questions and answers relating to the linked category:
1. Is Medical Cannabis Legal?
2. What is the Difference Between a Prescription and a Recommendation?
3. Which Physicians are Permitted to Issuer a Physician Certification to Recommend the Use of Medical Cannabis and How Do I Sign Up?
4. For What Conditions May I Issue a Physician Certification for the Use of Medical Cannabis?
5. What do I Need to do to Certify My Patient's Qualifying Condition?
6. How Do I Establish That I Have the Informed Written Consent of My Client?
7. Am I Subject to Dosage Limitations?
8. Must I Re-evaluate My Patients?
9. What if I am Already Making Recommendations of Low-THC and Medical Cannabis Under Current Law?
10. What Can Happen if I do Not Follow Applicable Federal and State Laws and Regulations?

View specific Questions and Answers related to each master category that follow. The answers collectively contain a narrative of the Qualified Physician's minimal legal and regulatory obligations.

Click on Physician Forum:

View introduction.

View Forum Search Bar. Insert search category text and click on search to be directed to a string of discussions regarding the category.

View the following Forum Categories: Specifically Approved Diseases and Conditions. Medical Conditions of the Same Kind or Class. Indicas, Sativas, and Hybrids. Recommended Dosages. Route of Administration Issues. Interactions, Side Effects and Contra-indications. Legal and Regulatory Issues.

Click on any category or execute a search command and you land on a page with all of that category's discussion threads.

A physician may start his or her own thread and become its moderator by selecting a category for the thread and posting in the dialog box.

A physician may join or respond to any existing thread by clicking on the thread, typing a response in the text box and electing whether or not he or she wants the author to be notified of the response via email.

Click on Upcoming Events.

View the type of category of available events. Insert text to search for a particular category; i.e., annual conference or patient marketing event.

View calendar. Events are to be listed on the calendar and can be added to scrolling

Alerts.

Click on Blogs, News and Updates. View desired Blog.

Click on “Read More” to see remainder of blog.

Click and insert text to leave a comment. Forward the blog if desired.

The invention is not limited to any specific state law or rule, and while Florida has been referenced in the examples, other state laws and rules can be utilized.

The system can be utilized to form a patient database stored in the non-volatile, non-transitory memory of the server. The patient database can include pertinent patient information regarding, among other things, the patient's medical cannabis experiences. The patient database can include typical information that would ordinarily be found in a patient's medical cannabis record, including but not limited to information relating to the condition(s) for which medical cannabis is recommended, side effects, effects of medical cannabis use on opioid use and/or addiction, other medications being taken by the patient, other medical conditions that the patient is suffering from, and efficacy of medical cannabis for the treatment of the patient's chronic condition. The patient database can be used for marketing and research.

The system can provide cannabis product growers, processors, and sellers with access to the system to communicate with recommending physicians and prospective patients, and can provide those seeking to reach out to Physician- Members and patients including but not limited to attorneys, insurance companies, electronic medical records and practice management compliance companies, and others with an additional means to market their products or services.

The system provides a medical practitioner database stored in the non-transitory tangible memory on the server, that contains information relating to medical practitioners.

The system also provides a medical cannabis database stored in the non-transitory tangible memory on the server, that contains information relating to, among other things, medical cannabis, including but not limited to, dosage, treatment, effects and com positions.

The system further provides a cannabis supplier database stored in the non-transitory tangible memory on the server, that contains information relating to medical cannabis growers, processors, and sellers.

The system also provides cannabis banking database stored in the non-transitory tangible memory on the server, that contains information relating to banks, lenders and other financial institutions that can conduct business with those businesses engaged in the medical cannabis industry where such activities are permitted under territorial, state or local law.

### EXAMPLE 2

### The Screen-Shots Shown in FIGS. 7, 8, 13, 14, And 17-29 Have Been Taken from A Working Website

FIGS. 16A-16C illustrate flow charts of an exemplary system and method, which will be described in reference to the attached Figures of exemplary screen-shots.

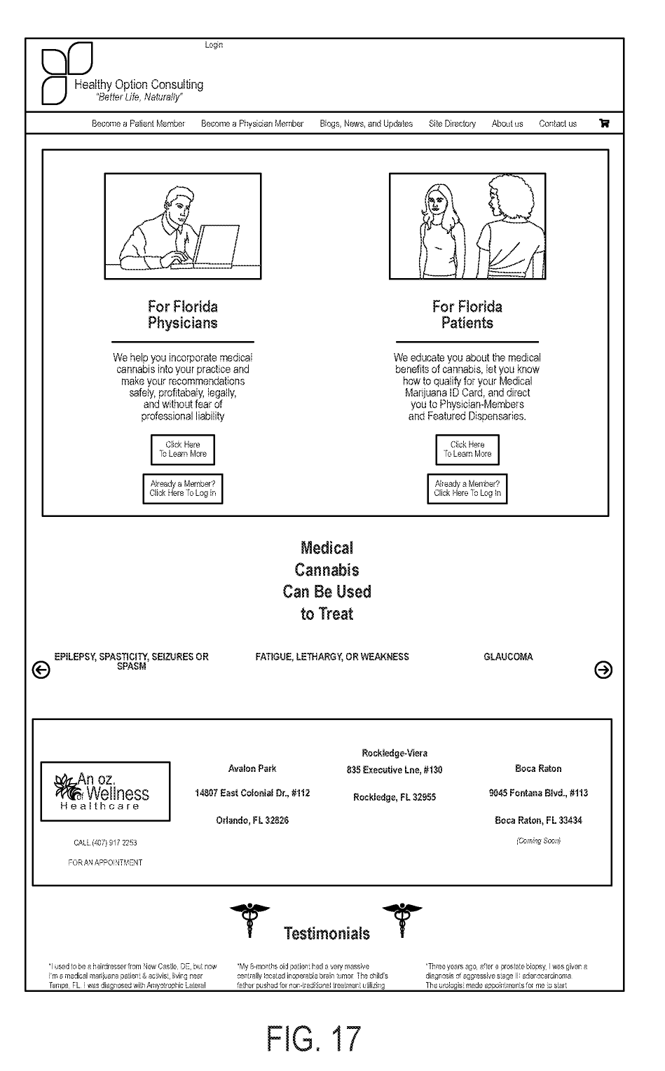

Medical practitioners, patients and prospective patients viewing the free front end homepage (FIG. 17) have the opportunity to view a brief description of the services provided to physicians and patients, the medical conditions for which medical cannabis can be used by patients in Florida, a list of the affiliated medical practices owned and/or managed by the Owner, testimonials offered by healthcare providers or patients who have benefited from the use of medical cannabis, and a scrolling Alert bar displaying additional messaging.

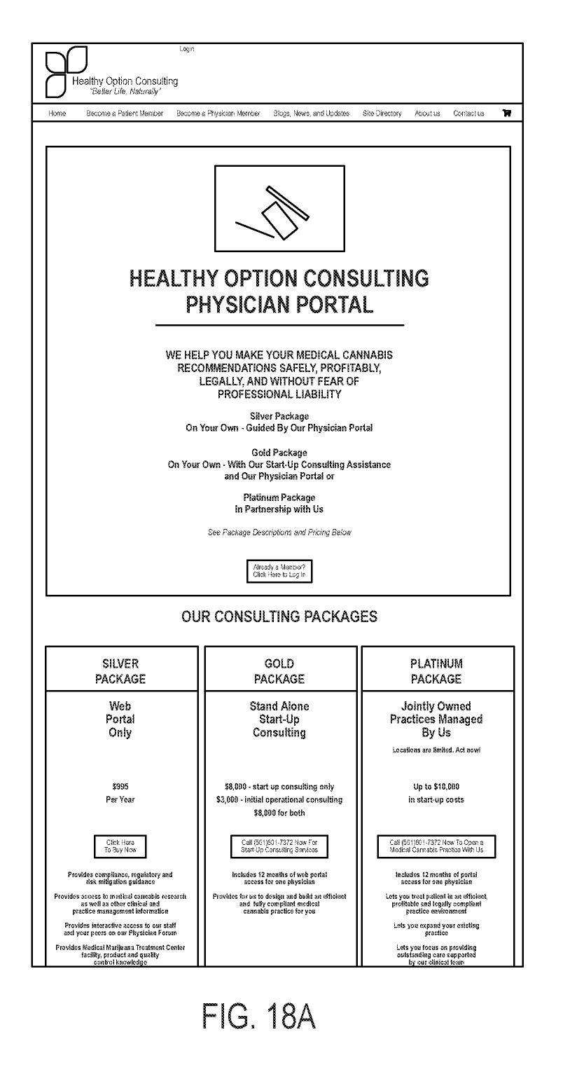

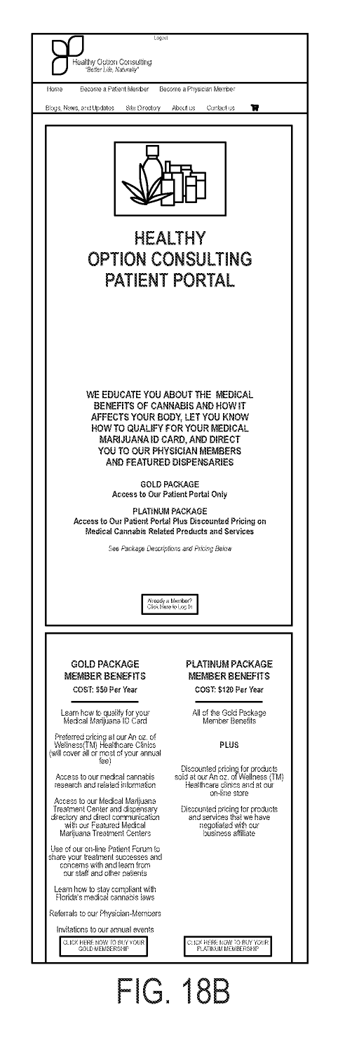

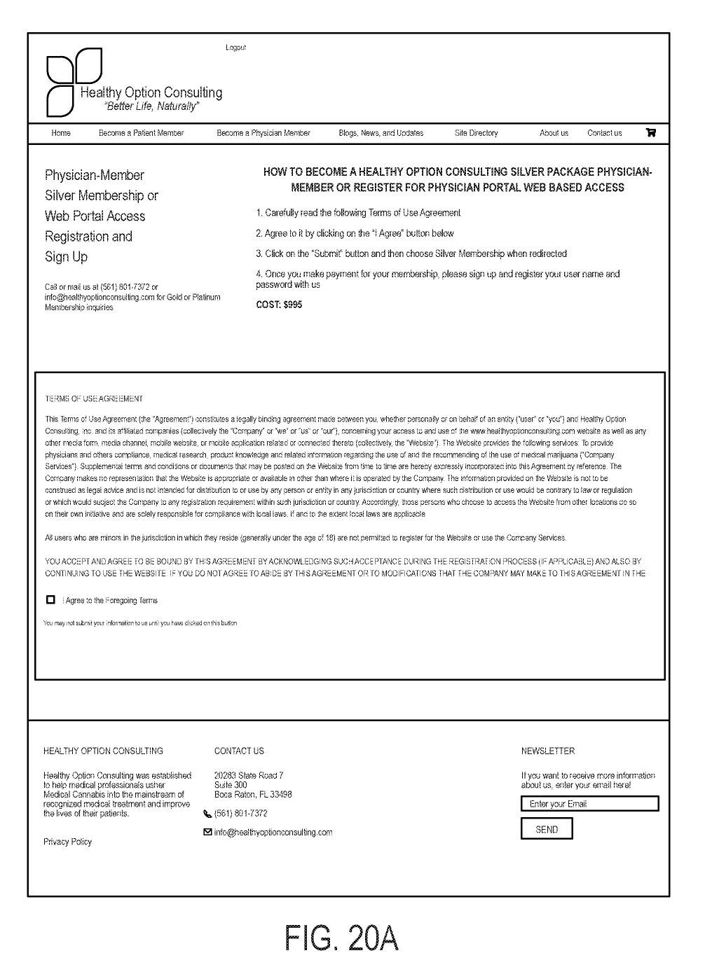

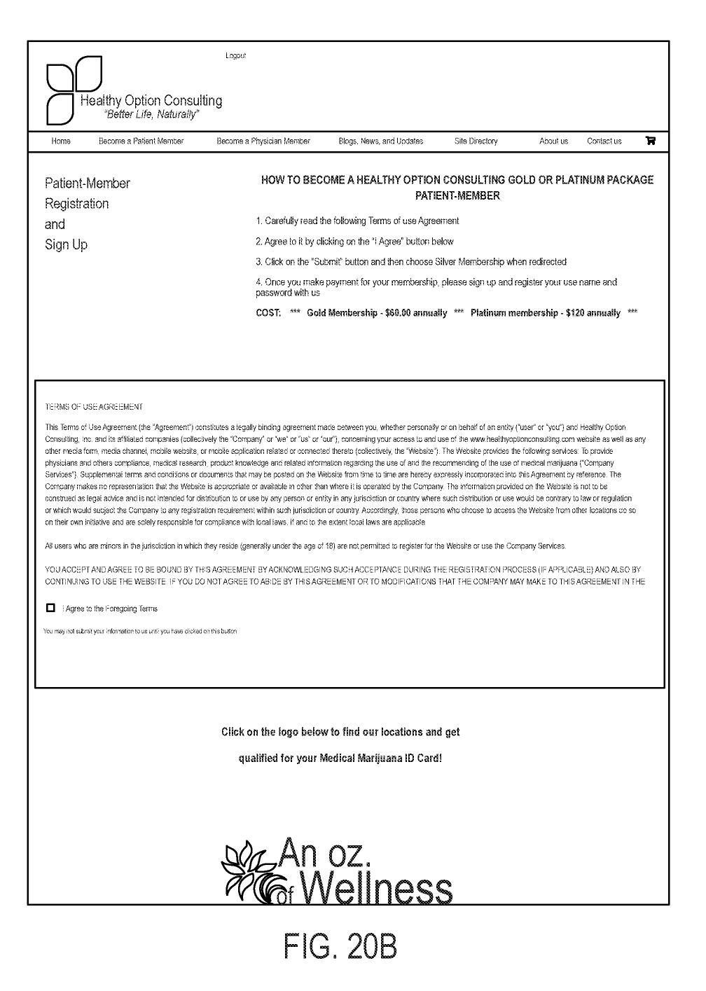

Medical practitioners seeking to learn more or to sign up with us a Physician-Member either click on the “Become a Physician Member” link on the header menu where they are directed to our Physician-Member Sign Up and Registration Page, FIG. 20A, or click on the “Learn More” button where they are directed to our Physician Portal Landing Page (FIG. 18A), where they can learn more about physician membership and the different consulting packages offered to physicians and then sign up to become a Physician-Member.

Patients seeking to learn more or to sign up with us a Patient-Member either click on the “Become a Patient Member” link on the header menu where they are directed to our Patient-Member Sign Up and Registration Page, FIG. 20B, or click on the “Learn More” button where they are directed to our Patient Portal Landing Page (FIG. 18B), where they can learn more about patient membership and the different membership packages offered to patients, or click on a link directing them to the affiliated medical practices owned and managed by the Owner, and then sign up with us to become a Patient-Member.

All pages on the website contain one or more links allowing physicians and patients to log on to the website and allow all viewers to forward questions or comments to the Owner.

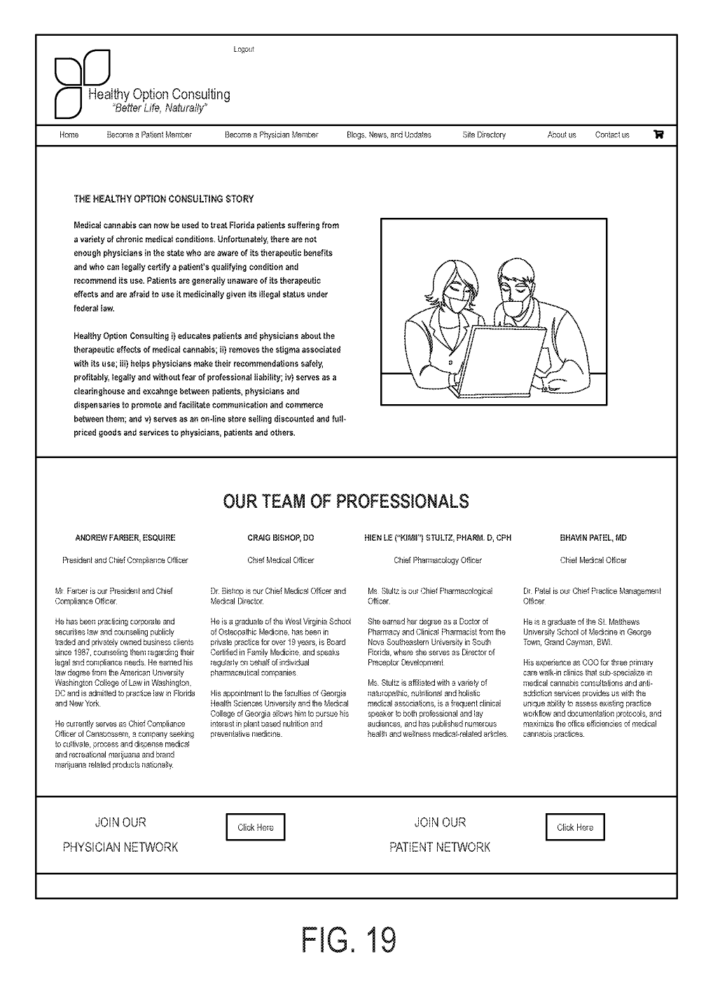

Sign Up—Physicians: To sign up with the system and become a Physician-Member, the user can click on the “Become a Physician Member” link on the website's header menu, the “Click Here To Buy Now” button appearing on the Physician Portal Landing Page (FIG. 18A), or the “Click Here” Button appearing on our “About Us” page (FIG. 19). After agreeing to our Terms of Use Agreement (FIG. 20A), registering and making payment, Physician-Members gain access to the private, paid-for portion of the website where they can, among other things, learn how to recommend medical cannabis to their patients safely, efficiently, profitably, legally and without fear of professional liability and how the system can drive medical cannabis patients to their practices.

Sign Up—Patients: To sign up with the system and become a Healthy Option Consulting Patient-Member, the user can click on the “Become a Patient Member” link on the website's header menu, the “Click Here Now . . . ” buttons appearing on the Patient Portal Landing Page (FIG. 18B), or the “Click Here” Buttons appearing on our “About Us” page (FIG. 19). After agreeing to our Terms of Use Agreement (FIG. 20B), registering and making payment, Patient-Members gain access to the private, paid for portion of our website where they can, among other things, learn about the medical benefits of cannabis, how to qualify for their Medical Marijuana ID Cards, how to qualify for discounted pricing for medical cannabis examination fees, and how to receive discounted pricing for other products and services.

Private access to the back end of our website teaches physicians how to incorporate medical cannabis into their practices, how to recommend medical cannabis to their patients safely, efficiently, profitably, legally and without fear of professional liability, and lets them know how we can direct medical cannabis patients to their practices.

Private access to the back end of our website teaches patients about the medical benefits of cannabis, how they can qualify for their Medical Marijuana ID Cards, how they can qualify for discounted pricing for medical cannabis examination fees, and how they can receive discounted pricing on other goods and services.

Once Physician-Members gain access to the private, back end of our website by registering with and paying us, they are directed to our Physician Site Directory (FIG. 7), which currently displays six links that they may click on, that bear references and link to the following modules which are designated as “Peer Reviewed and Anecdotal Medical Cannabis Research”, “Medical Marijuana Treatment Centers Product Knowledge”, “Our Patient Initiatives”, “Laws, Regulations, Compliance and Liability”, “Physician Forum”, and “Upcoming Events”.

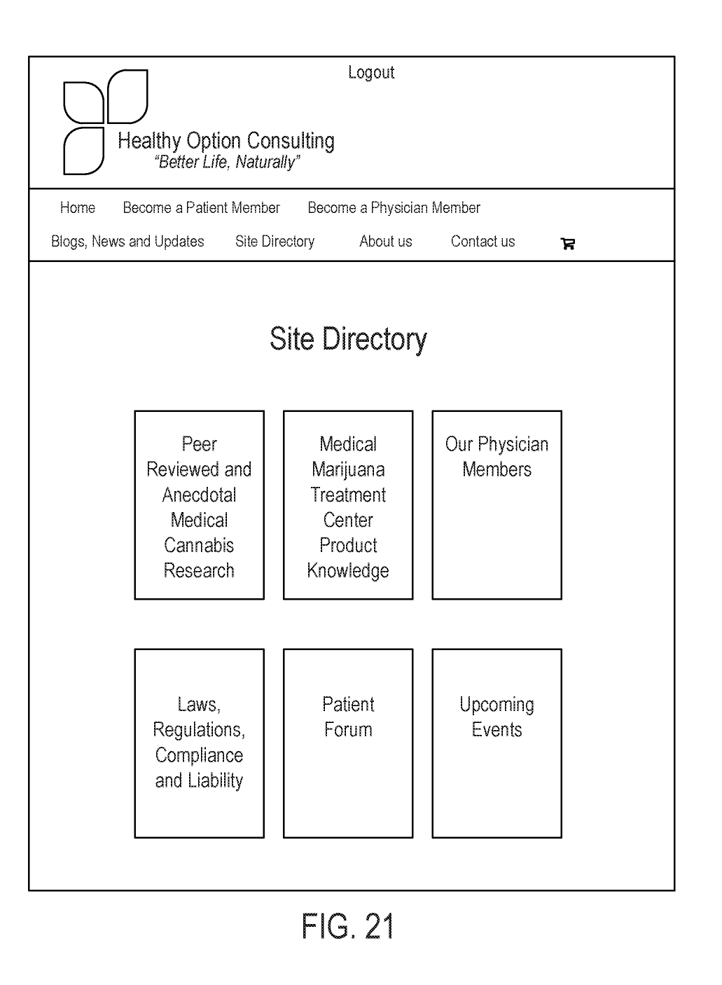

Once Patient-Members gain access to the private, back end of our website by registering with and paying us, they are directed to our Patient Site Directory, FIG. 21, which currently displays six links that they may click on, that bear references and link to the following modules which are designated as “Peer Reviewed and Anecdotal Medical Cannabis Research”, “Medical Marijuana Treatment Center Product Knowledge”, “Our Physician-Members”, “Laws, Regulations, Compliance and Liability”, “Patient Forum”, and “Upcoming Events”.

Peer Reviewed and Anecdotal Medical Cannabis Research—for Physicians (FIG. 8):, This module provides Physician-Members with the clinical tools needed to conduct their medical cannabis practices. It provides a catalogue of medical cannabis research articles that are generally categorized by the enumerated conditions for which medical cannabis may be used, symptoms and physiology. Each general category is then broken down into subcategories. As a user clicks on a subcategory, a page appears listing the research article(s) related to the subcategory. Physicians can then access our referenced medical articles by clicking on a pdf link to the extent one is available, or copying and pasting to an external site. This section also includes internal links to general medical cannabis related information and information relating to its metabolization, bioavailability, administration, effects, complications, contraindications, dosing, and best practices protocols.

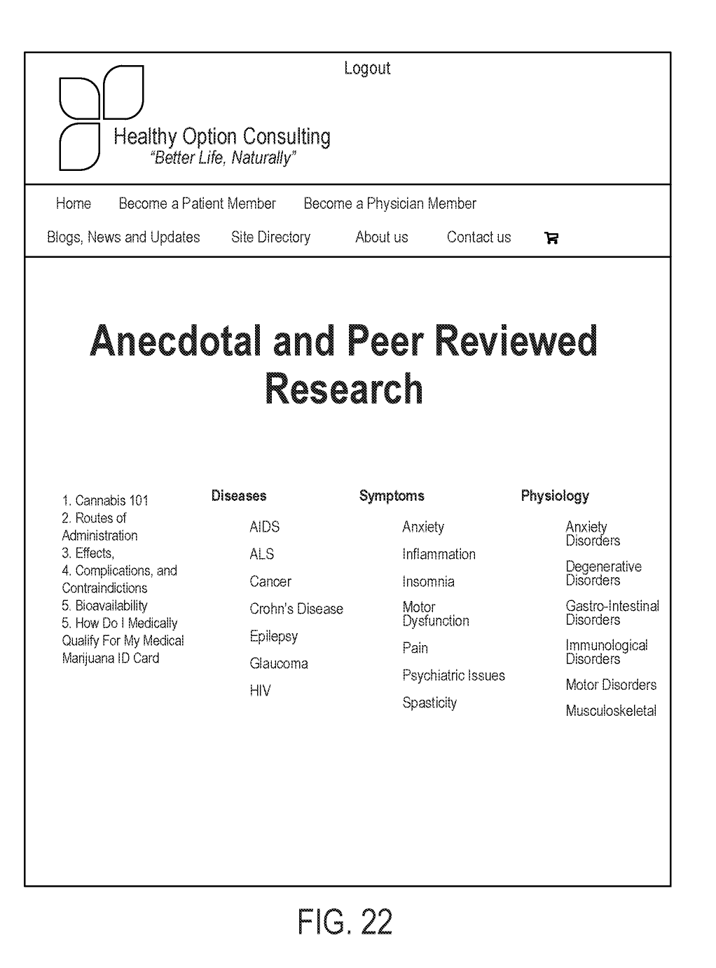

Peer Reviewed and Anecdotal Medical Cannabis Research—for Patients (FIG. 22): This module provides Patient-Members with the clinical and other information that they need to learn how medical cannabis can be used to treat their chronic medical conditions, and help them determine whether or not they qualify for their Medical Marijuana ID cards. It provides a catalogue of medical cannabis research articles that are generally categorized by Florida list of conditions for which medical cannabis may be used, symptoms and physiology. Each general category is then broken down into subcategories. As a user clicks on a subcategory, a page appears listing the research article(s) related to the subcategory. Patients can then access our referenced medical articles by clicking on a pdf link to the extent one is available, or copying and pasting to an external site. This section also includes internal links to general medical cannabis related information that is presented to Patient-Members in a manner more suited for lay audiences.

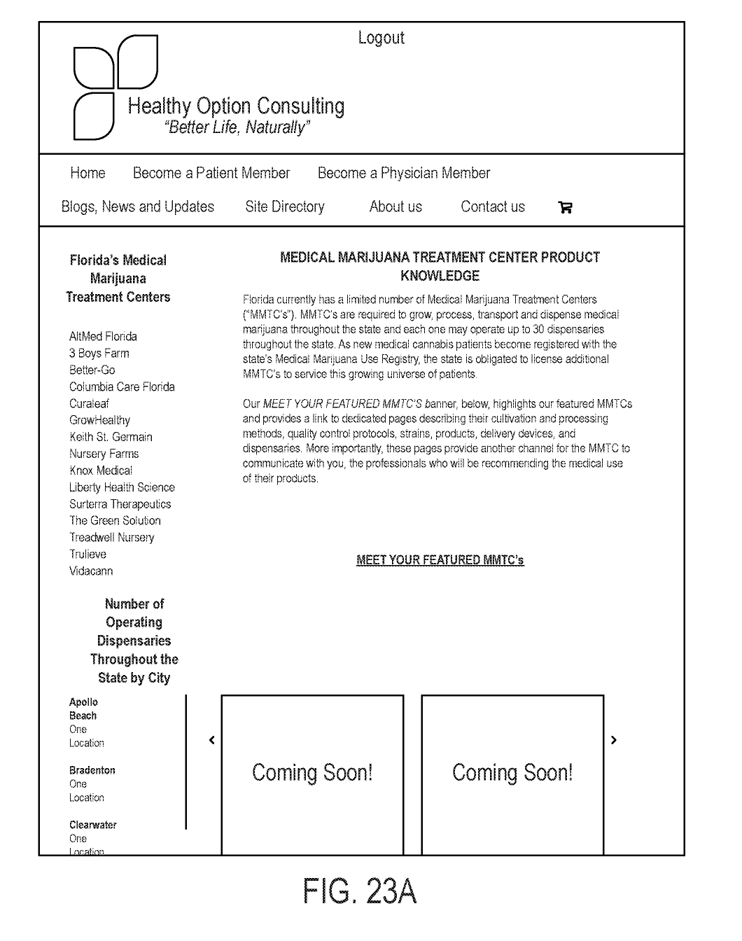

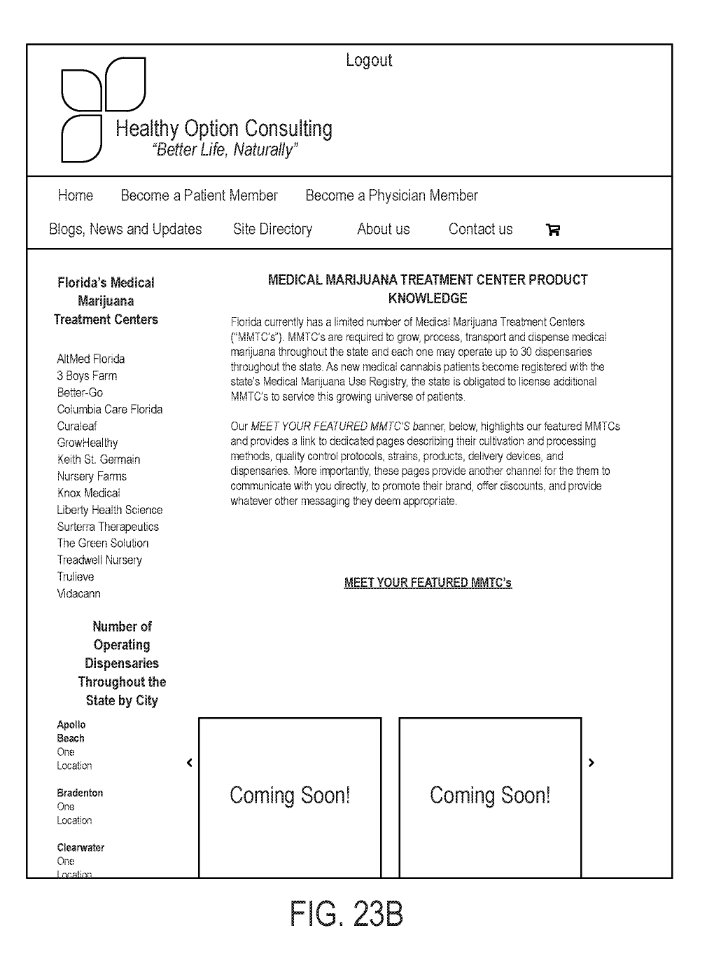

Medical Marijuana Treatment Centers Product Knowledge—for Physicians (FIG. 23A): This module provides Physician-Members with information regarding Florida's Medical Marijuana Treatment Centers. The left column on this page lists the Medical Marijuana Treatment Centers licensed to do business in Florida and the Florida cities within which operating dispensaries are currently doing business.

The remainder of this module provides links to our Featured Medical Marijuana Treatment Centers that are listed on our website for an annual fee. Each link allows a Medical Marijuana Treatment Center to educate physicians about its respective cultivation and processing methods, dispensaries, quality control protocols, product lines, delivery devices, and delivery services and present their desired branding, messaging and other information directly to our Physician-Members.

Medical Marijuana Treatment Centers Product Knowledge (for Patients), FIG. 23B: This module provides Patient-Members with information regarding Florida's Medical Cannabis Treatment Centers. The left column on this page lists the Medical Marijuana Treatment Centers licensed to do business in Florida and the Florida cities within which operating dispensaries are currently doing business.

The remainder of this module provides links to our Featured Medical Marijuana Treatment Centers that are listed on our website for an annual fee. Each link allows a Medical Marijuana Treatment Center to educate patients about its respective cultivation and processing methods, dispensaries, quality control protocols, product lines, delivery devices, and delivery services, and present their desired branding, messaging and other information directly to our Patient-Members.

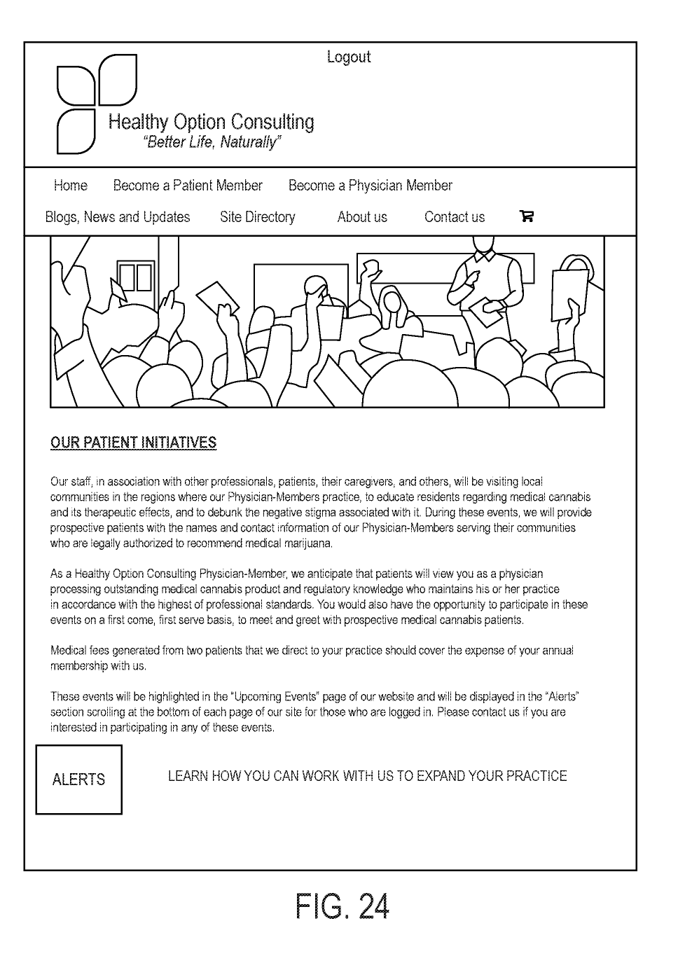

Our Patient Initiatives—for Physicians (FIG. 24): This module describes to Physician-Members how the Owner employs patient education and outreach initiatives to let the patient community at large learn about the benefits of medical cannabis and remove any stigma regarding its use. It describes how Physician-Members can participate in these programs to direct patients to their practices. It also describes how Patient-Members can be independently directed to their Practices.

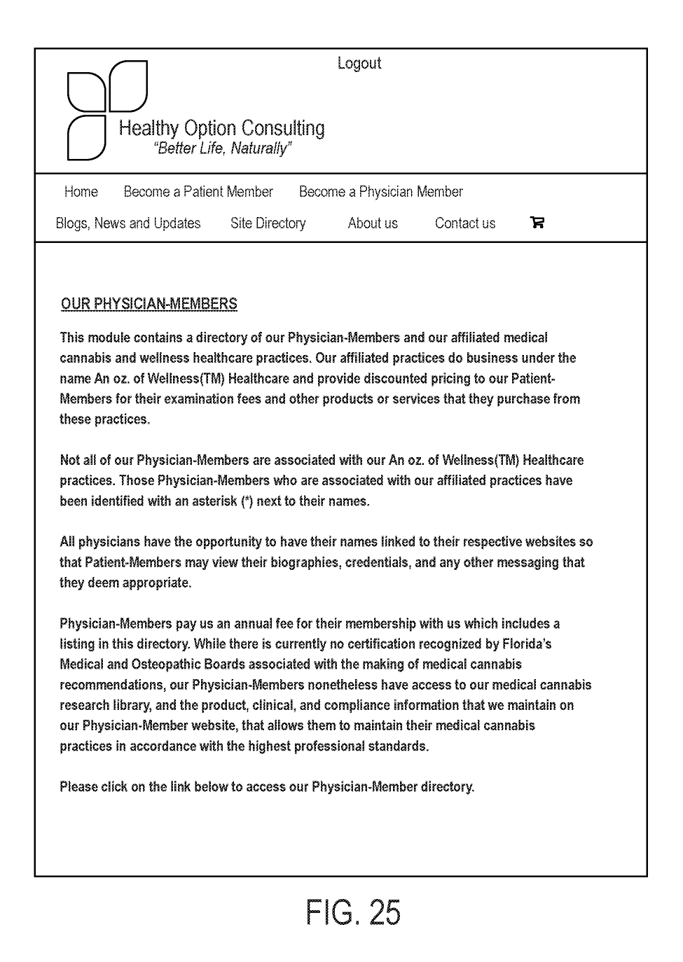

Our Physician-Members—for Patients (FIG. 25): This module provides our Patient-Members with a listing of our Physician Members, and links to their medical cannabis practices that enable our Physician-Members to directly communicate with out Patient-Members and present their unique messaging.

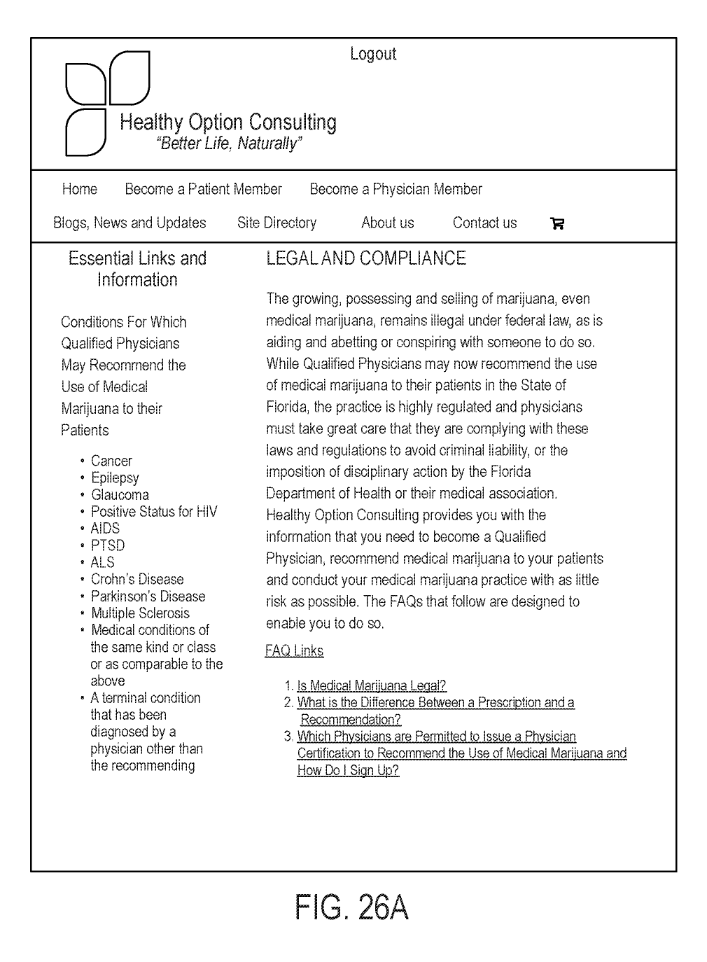

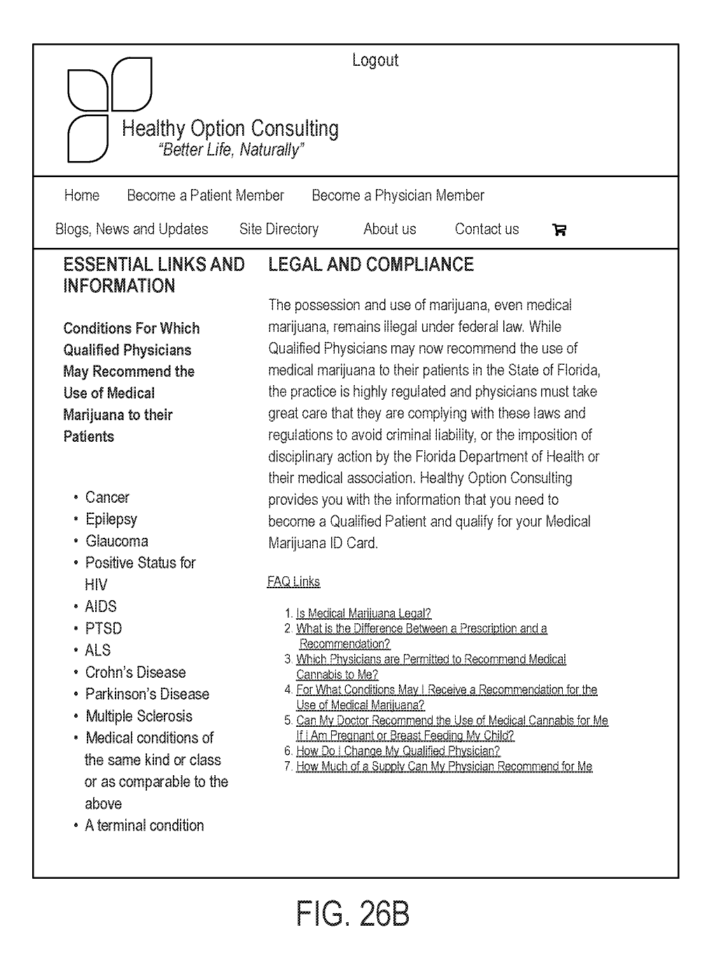

Laws, Regulations, Compliance and Liability—for Physicians (FIG. 26A): This module contains the information needed by Physician-Members to enable them to operate their medical cannabis practices in compliance with Florida and federal law, and with low risk of professional liability. This module currently serves as our on-line store and contains links to business affiliates who offer discounted or full priced products and services through the website.

This module is comprised of two columns. The left column contains Essential Links and Information, including a list of the conditions for which physicians may recommend the use of medical cannabis under Florida law, and an analysis of what the term “same kind or class or as comparable to” means. This analysis can be accessed by clicking on a link to a proprietary Classification Chart, which can be used to assist physicians with their determination as to what medical conditions are of the same kind or class or otherwise comparable to those specific conditions enumerated in Florida's state constitution and statutes. The remainder of the left column contains a listing of our business affiliates and store, and a link to the Florida law and constitutional provision enabling the use of medical cannabis by patients in Florida.

The right column contains information describing the compliance measures required of physicians seeking to recommend the use of medical cannabis to their patients along with an analysis of the application of federal and state laws to such recommendations.

The compliance related information displayed on this page is presented in the form of a series of frequently asked questions categorized into generalized topics. The issues raised in each of the generalized topics are addressed by a group of related questions and answers which, when viewed as a whole, and considered in conjunction with other information contained in the website, can enable the physician to make medical cannabis recommendations safely, with minimal legal risk, and with minimal risk of professional liability.

Laws, Regulations, Compliance and Liability—for Patients (FIG. 26B): This module contains the information needed by Patient-Members to help them determine whether they qualify for their Medical Marijuana ID Card and how to use their medical cannabis in accordance with Florida law. This section also currently serves as our on-line store and contains links to our business affiliates who offer discounted or full priced medical cannabis related products and services.

This module is comprised of two columns. The left column contains Essential Links and Information, including a list of the conditions for which physicians may recommend the use of medical cannabis under Florida law and an analysis of what the term “same kind or class or as comparable to” means. This analysis can be accessed by clicking on a link to a proprietary Classification Chart, which can be used to help patients determine whether their chronic medical conditions allow them to qualify for their Medical Marijuana ID card if they do not suffer from one of the enumerated conditions for which such use is specifically permitted. The remainder of the left column contains a listing of our business affiliates and our store, and a link to the Florida law and constitutional provision enabling the use of medical cannabis by patients in Florida.

The right column contains information describing those sections of Florida law governing the possession and use of medical cannabis by Florida patients and the sections governing patient caregivers.

The compliance related information displayed on this page is presented in the form of a series of frequently asked questions categorized into generalized topics. The issues raised in each of the generalized topics are addressed by a group of related questions and answers which, when viewed as a whole, and considered in conjunction with other information contained in the website, can help patients qualify for their Medical Marijuana ID Card, and enable patients and caregivers to possess and/or use medical cannabis in accordance with Florida law.

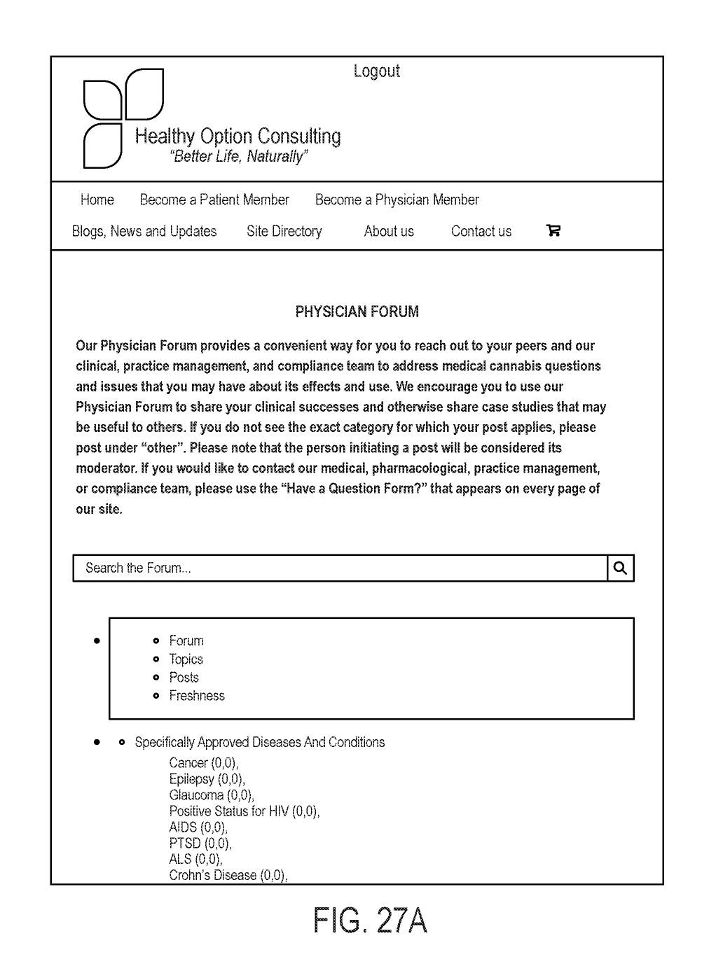

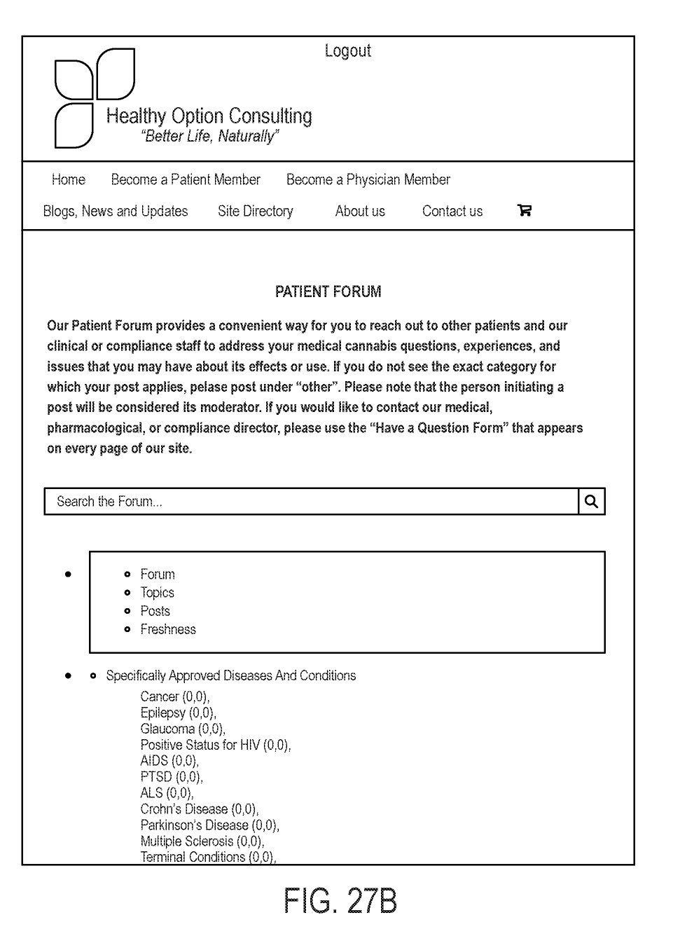

Physician Forum—for Physicians (FIG. 27A): This module contains an interactive on-line forum designed to enable our Physician-Members to reach out to their peers, the staff of the Owner, and others to address medical cannabis related questions and issues that they may have, to share their case studies and clinical successes, and to provide the Physician-Member community with their practice related insights. The Physician Forum is composed of multiple discussion categories and subcategories which can be readily expanded should the need arise. The user initiating a discussion is considered the thread's moderator, and by posting a question, concern or issue, solicits responses from the system and its community of users. A primary purpose of the Physician Forum is to lead to the overall improvement in the understanding of medical cannabis and its therapeutic effects by physicians and to help them make their medical cannabis recommendations, aid in the establishment of new medical cannabis clinical protocols, and improve the lives of patients.

Patient Forum—for Patients (FIG. 27B): This module contains an interactive on-line forum designed to enable Patient-Members to reach out to other patients, the staff of the Owner, and others to address medical cannabis questions and issues that they may have and to share their medical cannabis related experiences. The Patient Forum is composed of multiple discussion categories and subcategories which can be readily expanded should the need arise. The user initiating a discussion is considered the thread's moderator, and by posting a question, concern or issue, solicits responses from the system and its community of Patient-Members and other users. A primary purpose of the Patient Forum is to lead to the overall improvement in the understanding of medical cannabis and its therapeutic effects by patients and to remove any stigma regarding its use.

Upcoming Events—for both Physicians and Patients (FIG. 13): This module contains a calendar of all future events that the Owner and system will be sponsoring. It contains a search bar that can enable the user to search for a particular type of event. Upcoming community speaking engagements as well as annual conferences can be reflected in the Upcoming Events calendar and can also be featured in the scrolling Alerts appearing at the bottom of each page of the website.

Blogs, News and Updates—for both Physicians and Patients (FIG. 14): This module contains topical blogs authored by medical, pharmacological, practice management, and compliance staff of the Owner, concerning their respective specialties and interests. Physician and patient members may share these blog posts with others and leave comments for the author.

### Exemplary Website Map

Front End Function: Explains the system, and how the Owner can help physicians incorporate medical cannabis into their practices and make their recommendations safely, profitably, legally and without fear of professional liability, and how the Owner can educate patients about the medical benefits of cannabis, let them know how to qualify for their Medical Marijuana ID Card and directs them to Physician-Members and Featured Dispensaries. It provides for physicians and patients to click to learn more or log in if they are already members.

Get to Site by Clicking on web address, https://www.healthyoptionconsulting.com. View Home Page (FIG. 17) with header (Every page asks if you have a question so you can contact the Owner). View carousel of conditions for which medical cannabis can be used by patients in Florida. View the phone number and addresses of medical cannabis and wellness healthcare centers owned and/or managed by the Owner. View Testimonials-Click on “Read More” to see balance of each testimonial, and View Alerts.

Click About Us (FIG. 19) to Read About Management of the Owner.

Click Learn More to be Directed to the Physician Portal Landing Page (FIG. 18A) or the Patient Portal Landing Page (FIG. 18B)

View—A description of the services provided to physicians or patients, as the case may be, by the Owner and the pricing for all available consulting or membership packages.

View—The phone number appearing on the physician portal landing page to call us to purchase stand alone consulting services or to jointly open a medical cannabis practice.

Click to Contact Us to purchase stand alone consulting services or to jointly open a medical cannabis practice.

Click to Buy the Web-Based Only Physician Consulting Package.

Click to Buy Gold or Platinum Patient Membership

View—Physician Member Sign Up and Registration Page (FIG. 20A) or Patient-Member Sign-Up and Registration Page (FIG. 20B), as the case may be, Instructions for Registration and Sign Up, and Terms of Use Agreement.

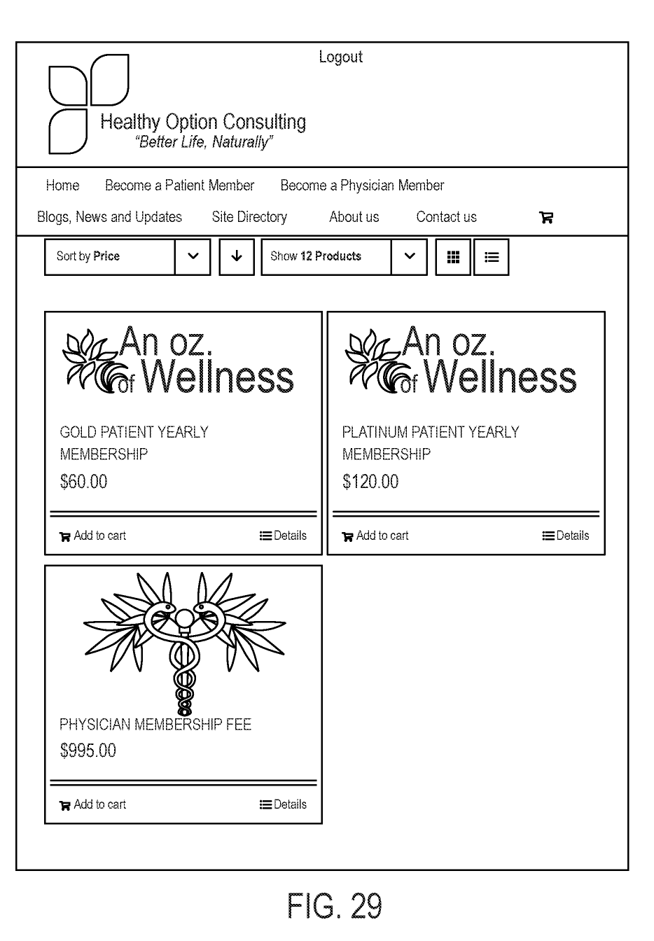

Click I Agree to the Foregoing Terms to purchase the type of membership desired, Add your information to register, Make Payment and Join. Choice of Membership is made on the Product Page of the Website (FIG. 29).

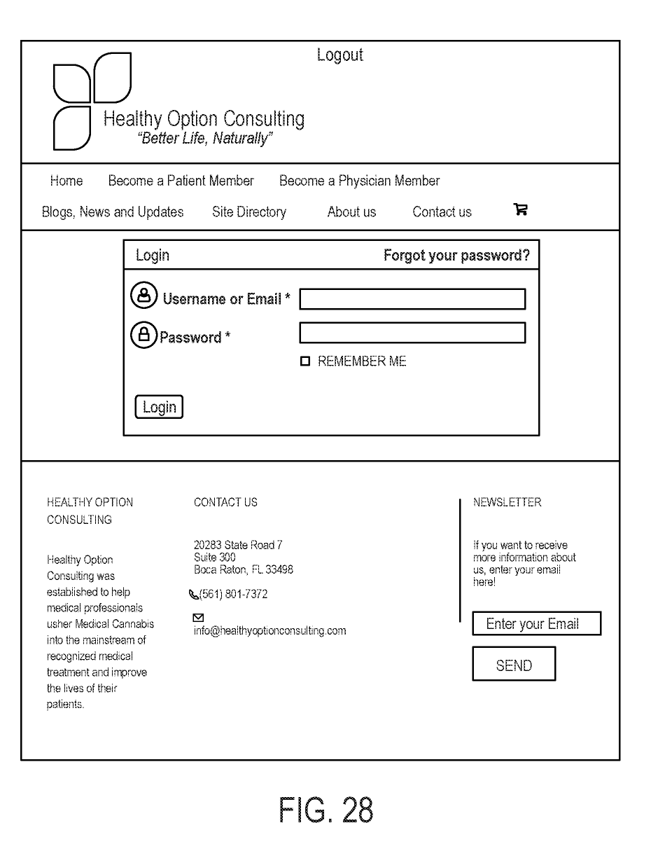

Click to Log In (FIG. 28).

Back End—for Physicians: Each page has a scrolling news Alert and “Have a Question?” link at the bottom and a header menu containing a link for Blogs, News and Updates and the Site Directory.

View Physician-Member Site Directory with its six linked and categorized buttons (FIG. 7).

Click on the appropriate button/link to see the desired portion of the website that you would like to view.

Click on Peer Reviewed and Anecdotal Medical Cannabis Research (FIG. 8): View columns of covered diseases. View columns of Symptoms. View columns of affected physiologies.

Click on each of the subcategories within the three columns and the user can be directed to a catalogue of medical cannabis research articles related to the linked category. If available, a copy of the article in pdf format can appear when the article link is clicked.

Click link on left column to reveal dosage and titration guidance.

Click link on left column to reveal drug interaction information.

Click link on left column to reveal contraindication information.

Click link on left column to reveal best practice guidance.

Click on Medical Marijuana Treatment Center Product Knowledge (FIG. 23A): View the Names of Medical Marijuana Treatment Centers on the left. View the number of statewide dispensaries by city on the left. View description of Meet Your Medical Marijuana Treatment Center feature on the right. View slides of featured Medical Marijuana Treatment Centers.

Click on featured Medical Cannabis Treatment Center to view information that Featured

Medical Marijuana Treatment Centers provide to be published on the website.

Click on Our Patient Initiatives (FIG. 24)—Describes how Owner intends to educate prospective patients regarding the health benefits of medical cannabis, Owner's company, Physician-Members and featured dispensaries. Further describes how these initiatives can direct prospective patients to the offices of Physician-Members, to featured Dispensaries, and get them to become Patient-Members.

Click on Laws, Regulations, Compliance and Liability (FIG. 26A)—The screen is comprised of a narrow column on left and a wider column on the right. View Conditions for Which Medical Cannabis May be Recommended on the left. View analysis of “same kind or class or as comparable to” on the left.

Click on link to Classification Chart (on the left): View Our Medical Cannabis Business

Affiliates and store on the left. View Florida law and constitutional provision enabling the use of medical cannabis in Florida on the left.

Click on link to Florida Constitution language (on the left).

View Introduction to Legal and Compliance section on the right. Legal and Compliance tutorial is set up as a series of Frequently Asked Questions (FAQs). View link of FAQ categories.

Click on link to FAQ master categories to be directed to questions and answers relating to the linked category:
1. Is Medical Cannabis Legal?
2. What is the Difference Between a Prescription and a Recommendation?
3. Which Physicians are Permitted to Issuer a Physician Certification to Recommend the Use of Medical Cannabis and How Do I Sign Up?
4. For What Conditions May I Issue a Physician Certification for the Use of Medical Cannabis?
5. What do I Need to do to Certify My Patient's Qualifying Condition?
6. How Do I Establish That I Have the Informed Written Consent of My Client?
7. Am I Subject to Dosage Limitations?
8. Must I Re-evaluate My Patients?
9. What if I am Already Making Recommendations of Low-THC and Medical Cannabis Under Current Law?
10. What Can Happen If I Do Not Follow Applicable Federal and State Laws and Regulations?

Questions and answers related to each master category follow. The answers collectively contain a narrative of the Qualified Physician's minimal compliance obligations.

Click on Physician Forum (FIG. 27A):

View introduction.

View Forum Search Bar. Insert search category text and click on search to be directed to a string of discussions regarding the category.

View the following Forum Categories: Specifically Approved Diseases and Conditions. Medical Conditions of the Same Kind or Class. Indicas, Sativas, and Hybrids. Recommended Dosages. Route of Administration Issues. Interactions, Side Effects and Contra-indications. Legal and Regulatory Issues.

Click on any category or execute a search command and you land on a page with all of that category's discussion threads.

A Physician-Member may start his or her own thread and become its moderator by selecting a category for the thread and posting in the dialog box.

A Physician-Member may join or respond to any existing thread by clicking on the thread, typing a response in the text box and electing whether or not he or she wants the author to be notified of the response via email.

Click on Upcoming Events (FIG. 13).

View the type of category of available events. Insert text to search for a particular category; i.e., annual conference or patient marketing event.

View calendar. Events are to be listed on the calendar and added to scrolling Alerts.

Click on Blogs, News and Updates (FIG. 14).

View desired Blog.

Click on “Read More” to see remainder of blog.

Click and insert text to leave a comment. Forward the blog if desired.

Back End for Patients: Each page has a scrolling news Alert and “Have a Question?” link at the bottom and a header menu containing a link for Blogs, News and Updates and the Site Directory.

View Patient-Member Site Directory with its six linked and categorized buttons (FIG. 21).

Click on the appropriate button/link to see the desired portion of the website that you would like to view.

Click on Peer Reviewed and Anecdotal Medical Cannabis Research (FIG. 22): View columns of covered diseases. View columns of Symptoms. View columns of affected physiologies.

Click on each of the subcategories within the three columns and the user can be directed to a catalogue of medical cannabis research articles related to the linked category. If available, a copy of the article in pdf format can appear when the article link is clicked.

Click link on left column to reveal basic cannabis related information.

Click link on left column to reveal routes of administration information.

Click link on left column to reveal effects information.

Click link on left column to reveal complications and contraindications information.

Click link on left column to reveal bioavailability information.

Click link on left column to reveal how one qualifies for his or her Medical Marijuana ID Card.

Click on Medical Marijuana Treatment Center Product Knowledge (FIG. 23B): View the names of Medical Marijuana Treatment Centers on the left. View the number of statewide dispensaries by city on the left. View description of Meet Your Featured Medical Marijuana Treatment Center feature on the right. View slides containing links to the Featured Medical Marijuana Treatment Centers.

Click on the Featured Medical Marijuana Treatment Center links to view content and links supplied to the Owner by the Featured Medical Marijuana Treatment Centers.

Click on Our Physician-Members (FIG. 25)—View a listing of our Physician-Members along with links to their respective medical cannabis practices.

Click on the Physician-Member links to be directed to their respective websites and unique messaging.

Click on Laws, Regulations, Compliance and Liability (FIG. 26B)—The screen is comprised of a narrow column on left and a wider column on the right. View Conditions for Which Medical Cannabis May be Recommended on the left. View analysis of “same kind or class or as comparable to” on the left.

Click on link to Classification Chart (on the left): View Our Medical Cannabis Business Affiliates and store on the left. View Florida law and constitutional provision enabling the use of medical cannabis in Florida on the left.

Click on link to Florida Constitution language (on the left). View Introduction to Legal and Compliance section on the right. Compliance tutorial is set up as a series of Frequently Asked Questions (FAQs). View linked FAQ categories.

Click on link to FAQ master categories to be directed to questions and answers relating to the linked category:
1. Is Medical Cannabis Legal?
2. What is the Difference Between a Prescription and a Recommendation?
3. Which Physicians are Permitted to Issuer a Physician Certification to Recommend the Use of Medical Cannabis and How Do I Sign Up?
4. For What Conditions May I Receive a Physician Certification for the Use of Medical Cannabis?
5. Can My Doctor Recommend the Use of Medical Cannabis for Me If I am Pregnant or Breast Feeding My Child?
6. How Do I Change My Qualified Physician?
7. How Much of a Supply Can My Physician Recommend for Mem and How Often Do I need to See My Physician?
8. What is a Caregiver; How Many Caregivers Can I Have; and Who Can Provide This Service For Me?
9. Do I Have to Pay My Caregiver?
10. What Can Happen If I Do Not Follow Applicable Federal and State Laws and Regulations?

Questions and answers related to each master category follow. The answers collectively contain a narrative of the patient's minimal compliance obligations.

Click on Patient Forum (FIG. 27B):

View introduction.

View Forum Search Bar. Insert search category text and click on search to be directed to a string of discussions regarding the category.

View the following Forum Categories: Specifically Approved Diseases and Conditions. Medical Conditions of the Same Kind or Class. Indicas, Sativas, and Hybrids. Recommended Dosages. Route of Administration Issues. Interactions, Side Effects and Contra-indications. Legal and Regulatory Issues.

Click on any category or execute a search command and you land on a page with all of that category's discussion threads.

A Patient-Member may start his or her own thread and become its moderator by selecting a category for the thread and posting in the dialog box.

A Patient-Member may join or respond to any existing thread by clicking on the thread, typing a response in the text box and electing whether or not he or she wants the author to be notified of the response via email.

Click on Upcoming Events (FIG. 13).

View the type of category of available events. Insert text to search for a particular category; i.e., annual conference or other patient event.

View calendar. Events are to be listed on the calendar and added to scrolling Alerts.

Click on Blogs, News and Updates (FIG. 14). View desired Blog. Click on “Read More” to see remainder of blog.

Click and insert text to leave a comment. Forward the blog if desired.

The invention is not limited to any specific state law or rule, and while Florida has been referenced in the examples, the invention may relate to other states and their laws and rules.

The invention is not limited to a system or method that simultaneously involves patients, physicians, and cannabis product growers, processors and/or dispensers, and may relate to a system or method involving: i) patients (or other cannabis product consumers) and cannabis product growers, processors and/or dispensers; ii) physicians and cannabis product growers, processors, and/or dispensers; and iii) patients and physicians.

The system can be utilized to form a patient database stored in the non-volatile, non-transitory memory of server. The patient database can include pertinent patient information regarding the patient's medical cannabis experiences. The patient database can include typical information that would ordinarily be found in a patient's medical cannabis record, including but not limited to information relating to the condition(s) for which medical cannabis is recommended, side effects, effects of medical cannabis use on opioid use and/or addiction, other medications being taken by the patient, other medical conditions that the patient is suffering from, the effect of medical cannabis use on the continuing need of other medication being taken by the patient, and efficacy of medical cannabis for the treatment of the patient's chronic condition. The patient database can be used for marketing and research.

The system can provide cannabis product growers, processors, and sellers with access to the system to communicate with recommending physicians and patients, and can provide those seeking to reach out to Physician-Members, Patient-Members, and cannabis product growers, processors and dispensers, including but not limited to attorneys, insurance companies, electronic medical records companies, practice management companies, and others with an additional means to market their products or services.

The system provides a medical practitioner database stored in the non-transitory tangible memory on the server, containing information relating to medical practitioners.

The system also provides a medical cannabis database stored in the non-transitory tangible memory on the server, containing information relating to, among other things, medical cannabis dosage, contraindications, treatment, effects and compositions.

The system further provides a cannabis supplier database stored in the non-transitory tangible memory on the server, that contains information relating to medical cannabis growers, processors, and sellers.

The system also provides a cannabis banking database stored in the non-transitory tangible memory on the server, containing information relating to banks, lenders and other financial institutions that can conduct business with the cannabis industry.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and can include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A computer-implemented method for providing medical cannabis comprising:
providing a system comprising a server having a processor and a non-transitory tangible memory in communication with the processor, the server is connected to a network, the server is configured to provide a web interface on a user interface device in communication with the server over the network, and the memory having stored therein a member medical practitioner database, member patient database, member dispensary database, medical practitioner information, patient information, and dispensary information;
the member medical practitioner database comprising a list of medical practitioners that are members of the system and contact information;
the member patient database comprising a list of the patients that are members of the system, contact information, and medical information;
the member dispensary database comprising a list of dispensaries that are members of the system, their contact information, and product information;
the medical practitioner information comprising at least one of cannabis education for the medical practitioner as to why the medical practitioner should recommend use of medical cannabis to a patient, how the medical practitioner can incorporate medical cannabis into a practice, how the medical practitioner can recommend use of medical cannabis safely, profitably, legally and without fear of professional liability, and how prospective medical cannabis patients can be directed to the medical practice,
the patient information comprising at least one of information relating to medical cannabis education and uses, dosing, administration instructions, and how a prospective patient can qualify to use medical cannabis, and
the dispensary information comprising name and contact information of a dispensary as well as a description and the availability of its products,
connecting, by a medical practitioner, a first user interface device over the network to the server so that the web interface is displayed on the first interface device;
sending the medical practitioner information, by the server, to the first user interface device;
connecting, by a patient, a second user interface device over the network to the server so that the web interface is displayed on the second user interface device;
sending patient information, by the server, to the second user interface device;
connecting, by a dispensary, a third user interface device over the network to the server so that the web interface is displayed on the third user interface device;
sending dispensary information by the server, to the first and second user interface devices; and
the patient receiving cannabis from a dispensary in compliance with state law.

2. The method according to claim 1, further comprising, by the server, transmitting to the first user interface device information relating to how the owner of the system can consult with the medical practitioner on a fee for services basis to design and build a medical cannabis practice for the medical practitioner and train the medical practitioner and staff to operate the medical cannabis practice in an safe, profitable and regulatory compliant manner.

3. The method according to claim 1, further comprising, by the server, transmitting to the first user interface device information relating to how the medical practitioner and the owner of the system can co-venture to develop, own and operate a medical cannabis practice, with the owner being responsible for the operations and management of the medical cannabis practice using, among other things, the method and system comprising the invention.

4. The method according to claim 1, further comprising, by the server, transmitting to the second user interface device information relating to at least one of medical cannabis, medical practitioners who make recommendations of medical cannabis in their respective communities, and dispensaries where the patient can purchase medical cannabis and related devices.

5. The method according to claim 1, wherein the system further comprises a lawyer database listing member lawyers that are members of the system and contact information of the member lawyers, and the method further comprising connecting to the server, by a lawyer, a fourth user interface device, and transmitting from the fourth user interface device to the server legal advice relating to cannabis.

6. The method according to claim 1, wherein the system further comprises an insurance database listing member insurance companies that are members of the system and contact information of the member insurance company, and the method further comprising connecting to the server, by a representative of a member insurance company, a fifth user interface device, and transmitting from the fifth user interface device to the server insurance information relating to cannabis.

7. The method according to claim 1, wherein the system further comprises an estate planner and creditor-proofing database listing member companies providing estate planning and creditor-proofing services that are members of the system and contact information of the member companies, and the method further comprising connecting to the server, by a representative of a estate planning and creditor-proofing company, a sixth user interface device, and transmitting from the sixth user interface device to the server estate planning and creditor-proofing information.

8. The method according to claim 1, wherein the system further comprises an entity database of entities doing business with or seeking to do business with medical cannabis practitioners, patients or dispensaries that are members of the system and contact information of the member entities, and the method further comprising connecting to the server, by a representative of the entity, a seventh user interface device, and transmitting from the seventh user interface device to the server entity information.

9. The method according to claim 8, wherein the entity information relates to providing other entities with access to the system to provide their goods and services to at least one of growers, processors, dispensers, patients or medical practitioners.

10. The method according to claim 1, wherein the patient database comprises information including symptom and treatment information.

11. The method according to claim 1, wherein the member patient database includes at least one of patient medical cannabis experiences, conditions for which medical cannabis is recommended and/or used, side effects, effects of medical cannabis use, medications being taken by the patient, drug interactions, other medical conditions that the patient is suffering from, effects of medical cannabis use on the patient's need for other medication that the patient is already taking, and efficacy of medical cannabis for the treatment of patient medical conditions.

12. A system for providing information relating to cannabis comprising:
a server having a processor and a non-transitory tangible memory in communication with the processor, the server is connected to a network, the server is configured to provide a web interface on a user interface device in communication with the server over the network, and the memory having stored therein a member medical practitioner database, member patient database, member dispensary database, medical practitioner information, patient information, and dispensary information;
the member medical practitioner database comprising a list of medical practitioners that are members of the system and contact information;
the member patient database comprising a list of the patients that are members of the system, contact information, and medical information;
the member dispensary database comprising a list of dispensaries that are members of the system, their contact information, and product information;
the medical practitioner information comprising at least one of cannabis education for the medical practitioner as to why the medical practitioner should recommend use of medical cannabis to a patient, how the medical practitioner can incorporate medical cannabis into a practice, how the medical practitioner can recommend use of medical cannabis safely, profitably, legally and without fear of professional liability, and how prospective medical cannabis patients can be directed to the medical practice,
the patient information comprising at least one of information relating to medical cannabis education and uses, dosing, administration instructions, and how a prospective patient can qualify to use medical cannabis, and
the dispensary information comprising name and contact information of a dispensary as well as a description and the availability of its products; and
a user interface device constructed to connect to the server over the network.

13. The system according to claim 12, further comprising a dispensary connected to the server by the user interface device.

14. The system according to claim 12, further comprising a lawyer database listing member lawyers that are members of the system and contact information of the member lawyers.

15. The system according to claim 12, further comprising an insurance database listing member insurance companies that are members of the system and contact information of the member insurance company.

16. The system according to claim 12, further comprising an estate planner and creditor-proofing database listing member companies providing estate planning and creditor-proofing services that are members of the system and contact information of the member companies.

17. The system according to claim 12, further comprising an entity database of entities doing business with or seeking to do business with medical cannabis practitioners, patients or dispensaries that are members of the system and contact information of the member entities.

18. The system according to claim 17, wherein the entity information relates to providing other entities with access to the system to provide their goods and services to at least one of growers, processors, dispensers, patients or medical practitioners.

19. The system according to claim 12, wherein the patient database comprises information including symptom and treatment information.

20. The system according to claim 12, wherein the member patient database includes at least one of patient medical cannabis experiences, conditions for which medical cannabis is recommended and/or used, side effects, effects of medical cannabis use, medications being taken by the patient, drug interactions, other medical conditions that the patient is suffering from, effects of medical cannabis use on the patients continuing need for other medication that the patient is already taking, and efficacy of medical cannabis for the treatment of patient medical conditions.

21. A computer readable medium storing instructions in a non-transitory memory executable by the computer, wherein execution in the instructions implements a method according to claim 1.
